SEQUENCE LISTING (16 sequences)

SEQ ID NO: 1 - DNA, 22 bp, Artificial
  5′-primer for PCR
tgataagtgttggacttttagc

SEQ ID NO: 2 - DNA, 22 bp, Artificial
  3′-primer for PCR
cttcagttgataggtaacctgg

SEQ ID NO: 3 - DNA, 22 bp, Artificial
  5′-primer for PCR
ctgcagccattgttctttctac

SEQ ID NO: 4 - DNA, 23 bp, Artificial
  3′-primer for PCR
gtatcgataagctttcacacacc

SEQ ID NO: 5 - DNA, 24 bp, Artificial
  5′-primer for PCR
ataggatcctgctcaggcgagaga

SEQ ID NO: 6 - DNA, 24 bp, Artificial
  3′-primer for PCR
gacaagctttcacacaccaatgac

SEQ ID NO: 7 - protein, 8 aa, Artificial
  FLAG epitope at the N-terminus of pCMV-Tag expression vector
DYKDDDDK

SEQ ID NO: 8 - DNA, 22 bp, Artificial
  forward primer for PCR
tgataagtgttggacttttagc

SEQ ID NO: 9 - DNA, 22 bp, Artificial
  reverse primer for PCR
cttcagttgataggtaacctgg

SEQ ID NO: 10 - DNA, 21 bp, Artificial
  forward primer for PCR
ctagcgccaccatggctcagg

SEQ ID NO: 11 - DNA, 23 bp, Artificial
  reverse primer for PCR
cttctgtgacacacacggctgtg

SEQ ID NO: 12 - DNA, 28 bp, Artificial
  forward primer
gagctagcgccaccatggctcaggcgag

SEQ ID NO: 13 - DNA, 30 bp, Artificial
  reverse primer
tccgaattctcacacaccaatgactctttc

SEQ ID NO: 14 - DNA, 21 bp, Artificial
  forward primer for detection of CHO Beta-actin
agctgagagggaaattgtgcg

SEQ ID NO: 15 - DNA, 18 bp, Artificial
  reverse primer for detection of CHO Beta-actin
gcaacggaaccgctcatt

SEQ ID NO: 16 - protein, 15 aa, Artificial
  Peptide corresponding to the C-terminus region of the hamster CMP-sialic acid transporter (SwissProt accession number O08520)
CIQQEATSKERVIGV

(19) United States
(12) Patent Application Publication
(10) Pub. No.: US 2009/0298120 A1
(43) Pub. Date: Dec. 3, 2009

(54) METHOD FOR EXPRESSING SIALYLATED GLYCOPROTEINS IN MAMMALIAN CELLS AND CELLS THEREOF

(75) Inventors: Soo Ching Niki Cecilia Wong, Singapore (SG); Gek Sim Miranda Yap, Singapore (SG); I-Chyau Daniel Wang, Newton, MA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignees: NATIONAL UNIVERSITY OF SINGAPORE, Singapore (SG); MASSACHUSETTS INSTITUTE OF TECHNOLOGY, Cambridge, MA (US)

(21) Appl. No.: 11/568,591

(22) PCT Filed: May 4, 2005
(86) PCT No.: PCT/SG05/00139

§ 371 Date: Nov. 2, 2006

Related U.S. Application Data
(60) Provisional application No. 60/567,458, filed on May 4, 2004.

(30) Foreign Application Priority Data
May 4, 2004 (US) 60567458

(51) Int. Cl. C12P 21/02; C12N 5/06; C12N 5/08; C12N 5/10

(52) U.S. Cl. 435/69.1; 435/325; 435/358; 435/366; 435/375

(57) ABSTRACT

Methods and systems for producing glycoproteins having sialylated oligosaccharides are provided. The invention comprises the genetic engineered cells with a CMP-sialic acid transporter (CMP-SAT) gene so that the cells express the CMP-SAT protein or fragment thereof at an above endogenous levels. The increase in CMP-SAT expression allows for the increased transport of the CMP-sialic acid into the Golgi apparatus so as to obtain sialylation of glycoproteins at above endogenous levels. In particular, the methods and systems of the invention are useful for producing complex sialylated glycoproteins in mammalian cells of interest, for example Chinese Hamster Ovary (CHO) cells.

### REFERENCE TO RELATED APPLICATIONS

The present application claims priority from U.S. Provisional Patent Application No. 60/567,458, filed on May 4, 2004, the disclosure of which is incorporated herein in its entirety.

### FIELD OF THE INVENTION

The present invention relates to methods and systems for expressing sialylated glycoproteins in mammalian cells.

### BACKGROUND OF THE INVENTION

A recent survey of the SwissProt protein database predicted that more than half of all eukaryotic proteins are glycoproteins, and 90% of these are likely to contain N-linked glycosylation (Apweiler et al., 1999). Many of these glycoproteins are produced as recombinant products in mammalian cells for therapeutic applications (Andersen & Krummen, 2002). It is known that glycosylation affects critical properties of the glycoprotein such as its solubility, thermal stability and bioactivity (Jenkins & Curling, 1994). In particular, the presence of terminal sugar sialic acid increases the in vivo circulatory half life of glycoproteins as sialic acid terminated glycans are not recognized by asialoglycoprotein receptors (Weiss & Ashwell, 1989), which otherwise target glycoproteins for degradation. Hence, one of the goals of recombinant glycoprotein production is to achieve maximum and consistent sialylation on these recombinant glycoproteins.

Glycosylation occurs as a series of enzyme catalysed reactions in the ER and Golgi apparatus (reviewed in Kornfeld and Kornfeld, 1985; Varki, 1993). The nucleotide sugars, which serve as co-substrates in the reactions, are synthesized in the cytosol and are impermeable to the microsomal membranes. Nucleotide sugar transporter proteins thus exist to translocate the nucleotide sugars from the cytosol to the lumen (Hirschberg & Snider, 1987). As an example, the interplay of the various proteins involved in the terminal sialylation step is shown in FIG. 7 of the present patent application. Glycoprotein heterogeneity results from variation in different parts of this complex process. Glycosylation engineering approaches that have been employed to modify glycosylation profiles involve the manipulation of glycosyltransferases (reviewed in Bailey et al., 1998; Grabenhorst et al., 1999) and glycosidases (reviewed in Warner, 1999). Genetic manipulation of the host glycosylation pathway has been carried out to generate glycoform distributions that are more predictable and consistent. One such area of glycosylation engineering involves the manipulation of glycosylation patterns of existing glycoproteins by making mutations in their polypeptide chain to add oligosaccharides (Koury, 2003) or by mutating the positions of oligosaccharides (Keyt et al., 1994) to produce more efficacious proteins. Specific intervention of the host glycosylation pathway can be performed through the introduction or overexpression of glycosyltransferase genes (Fukuta et al., 2000; Sburlati et al., 1998) or antisense inhibition of endogeneous glycosylation genes (Ferrari et al., 1998) in the host cells.

The availability of nucleotide sugar substrates and the transport of these proteins through the ER and Golgi are also important determinants of the extent of protein glycosylation (Hooker et al., 1999). Various groups have attempted to overexpress sialyltransferases to improve sialylation. Chinese hamster ovary cells contain α2,3-sialyltransferase but not α2,6-sialyltransferase (Lee et al., 1989), resulting in glycoproteins which contain only α-2,3 linked sialic acids. However, human glycoproteins contain both α2,3 and α2,6 linked sialic acids. Many groups have thus attempted to over express α2,6-sialyltransferase to overcome this deficiency in α2,6 linked sialic acids (Minch et al., 1995; Lee et al., 1989). In addition, many groups have over expressed α2,6-sialyltransferase (Bragonzi et al., 2000; Jassal et al., 2001) and/or α2,3-sialyltransferase (Fukuta et al., 2000; Weikert et al., 1999) in attempts to improve sialylation of the recombinant protein. This has led to varying results, as summarized in Table 3 of the present application.

In addition, some groups have attempted to improve sialylation of the recombinant protein through sialic acid precursor (N-acetylmannosamine) feeding (Table 3 of the present application). N-acetylmannosamine (ManNAc) has been known to be a specific precursor for increasing intracellular sialic acid pools (Pels Rijcken et al., 1995). It was reported that step-wise increments in ManNAc feeding of up to a concentration of 20 mM to Chinese hamster ovary cells producing recombinant human interferon-gamma (CHO IFN-γ) increased intracellular sialic acid concentration. This in turn led to a 15% improvement in the sialylation of interferon-gamma (IFN-γ) (Gu and Wang, 1998). However, saturation in sialylation improvement was observed with the addition of 40 mM ManNAc. More interestingly, up to a 12-fold increase in intracellular sialic acid concentration was observed when 20 mM ManNAc was fed to NS0 cells producing a recombinant humanized IgG1 (Hills et al., 2001), as well as to both CHO and NS0 cells producing TIMP-I (Baker et al., 2001), with no apparent increase in recombinant protein sialylation.

Cytidine monophosphate-sialic acid (CMP-SA) must be delivered into the Golgi apparatus in order for sialylation to occur, and this transport process depends on the presence of the cytidine monophosphate-sialic acid transporter (CMP-SAT) (Deutscher et al. (1984) Cell 39:295-299). The CMP-sialic acid transporters (CMP-SATs) belong to the family of nucleotide sugar transporters. Similar to the other nucleotide sugar transporters, CMP-SATs are integral transmembrane proteins that reside on the Golgi membrane, where CMP-SA is transported into the Golgi via an antiport mechanism, as shown in FIG. 7 of the present patent application (reviewed in Berninsone & Hirschberg, 2000; Hirschberg et al., 1998; Hirschberg & Snider, 1987; Kawakita et al., 1998). Betenbaugh and others (PCT patent application published with the number WO01/42492) disclosed that human sialic acid synthetase increased sialic acid production in insect cells, which led to improvement in sialylation of proteins. Further, Betenbaugh and others (US patent application published with the number US2002/0065404 A1; and WO01/42492) speculated about the possibility of enhancing the expression of Drosophila CMP-SAT to increase the presence of CMP-SA into the Golgi apparatus and hence enhance sialylation of glycoproteins in insect cells.

The CMP-SAT gene sequence disclosed in US 2002/0065404 A1 and WO01/42492 was searched under the NCBI GenBank database to reveal a 100% match with 2 GenBank entries:
1) AF397530-Drosophila melanogaster CMP-sialic acid/UDP-galactose transporter mRNA, complete cds; and
2) AB055493-Drosophila melanogaster ugt mRNA for UDP-galactose transporter complete cds.

The first GenBank entry was a direct submission by Betenbaugh et al. (20 Mar. 2002) and the second was a direct submission by Segawa et al. (15 Jan. 2003).

Segawa et al. (2000) reported that where the CMP-SAT gene was cloned and expressed, it was experimentally demonstrated that UDP-galactose was transported, not CMP-sialic acid. In addition, they found that the encoded nucleotide sugar transporter also transported UDP-N-acetyl-galactosamine. Aumiller and Jarvis, 2002, disclosed that the CMP-SAT gene sequence was obtained through a homology search and was found to be similar to what was obtained by Betenbaugh et al. They subsequently cloned the nucleotide sugar transporter gene, and found through a genetic complementation assay and a biochemical assay that the protein encoded by the sugar transporter gene transports UDP-galactose, not CMP-sialic acid. Therefore, the Drosophila gene that was cited in US 2002/0065404 A1 and WO01/42492, which was supposed to be the CMP-SAT gene, turned out to be instead the gene encoding for the UDP-galactose transporter. Accordingly, US 2002/0065404 A1 and WO01/42492 do not provide any experimental proof or any other evidence that the CMP-SAT transports CMP-SA. On the contrary, Betenbaugh and others expressed UDP-galactose tranporter, which transports into the Golgi UDP-Galactose, not CMP-SA.

There is therefore a need in the art for further investigations as to whether the CMP-sialic acid transport system can be regulated so as to influence the amount of CMP-SA being transported into the Golgi. There is also a need in the art for alternative systems for the production of efficiently sialylated glycoproteins.

### SUMMARY OF THE INVENTION

The present invention addresses the problems above, and in particular provides an efficient method and system for producing sialylated glycoproteins, which mitigates the forgoing problems, or one which at least provides the public with a useful choice. In particular, the present invention provides for a method for the preparation of sialylated glycoprotein(s) of interest in a cell or cell line comprising enhancing the expression of a CMP-SAT, a fragment or a variant thereof, at above endogenous level. The present inventors have surprisingly found that enhancing the production of CMP-SAT at above the endogenous level improved the production of sialylated glycoproteins. In particular, the method of the invention is an efficient method for maximizing the sialic content of glycoproteins on interest, The maximum sialic acid content of glycoproteins is defined as the maximum number of moles of sialic acid that can be attached per mole of the glycoprotein based on the availability of sialic acid acceptor sites. In particular, the sialic acid acceptor sites for eukaryotic cells are glycans which terminate with β1,-4 galactose. The availability of β1,4-galactose sites in turn depends on the extent of prior glycoprotein processing and the glycan site occupancy of the glycoprotein. The methods and systems of the invention are useful for producing complex sialylated glycoproteins in mammalian cells of interest including, but not limited to, CHO cells.

According to a first aspect, the present invention provides at least a mammalian cell producing a CMP-SAT, a fragment or a variant thereof, at above endogenous levels. The mammalian cell may be any suitable mammalian cell for the purposes of the present invention, for example a human or CHO cell.

The invention also provides a cell line, for example in the form of a cell culture comprising the cell producing a CMP-SAT at above endogenous levels.

The invention comprises the genetic engineering of cells with a CMP-sialic acid transporter (CMP-SAT) gene so that the cell expresses the CMP-SAT protein at a level above the endogenous level. The increase in CMP-SAT expression allows for the increased transport of the CMP-sialic acid (CMP-SA) into the Golgi apparatus in order to obtain sialylation of glycoprotein(s) at above endogenous levels. Accordingly, it is provided at least a mammalian cell, wherein the cell is transformed with a gene encoding CMP-SAT or a construct comprising that gene. In particular, the mammalian cell is transformed with a mammalian CMP-SAT gene. For example, with a human or CHO CMP-SAT gene.

According to one aspect of the invention, the increase in sialylation is achieved by expressing a CMP-SAT protein, or a fragment or variant thereof, in the cell of interest. Accordingly, the invention provides at least one mammalian cell, wherein the cell produces at least one sialylated glycoprotein at above endogenous levels. In particular, the mammalian cell produces a CMP-SAT, a fragment or a variant thereof, at above endogenous levels, and at least one sialylated glycoprotein at above endogenous levels. The at least one glycoprotein may be heterologous. The glycoprotein may be mammalian, for example human or CHO glycoprotein. However, any other glycoprotein useful for the purposes of the present invention may be produced. In particular, the cell of the invention may be used for the production of a complex or mixture of sialylated glycoproteins of interest.

More in particular, the glycoprotein of interest may be any IFN-γ, a fragment or a variant thereof.

Accordingly, the mammalian cell or cell line of the invention may be transformed with a gene encoding a heterologous protein or with a construct comprising that gene. For example, the cell is transformed with a gene encoding at least an IFN-γ, a fragment or a variant thereof.

In particular, the mammalian cell or cell line of the invention may be a CHO cell, and the cell produces CMP-SAT, a fragment or a variant thereof, at above endogenous levels, wherein the cell produces at least one sialylated glycoprotein at above endogenous levels. The sialylated glycoprotein may be at least IFN-γ, a fragment or a variant thereof.

The mammalian cell of the invention may be an isolated cell line.

According to another aspect, the present invention provides a kit for the expression of at least one sialylated glycoprotein comprising the mammalian cell or cell line of the invention.

According to another aspect, methods and systems for producing glycoproteins having sialylated oligosaccharides are provided. Accordingly, the invention provides a method for the preparation of sialylated glycoprotein(s) in a cell or cell line comprising enhancing the expression of a CMP-SAT, a fragment or a variant thereof, at above endogenous levels. According to a further aspect, the sialylated glycoprotein(s) is also expressed at above endogenous levels. More in particular, the overexpression of CMP-SAT brings about a maximum sialylation of glycoprotein(s).

In particular, the mammalian cell or cell line may be CHO or human cell line. The sialylated glycoprotein(s) may be a heterologous mammalian glycoprotein. For example, the sialylated glycoprotein(s) is any IFN-γ, a fragment or a variant thereof.

According to another aspect, the invention provides a method for producing at least a sialylated glycoprotein in a mammalian cell or cell line comprising the steps of:
(a) transforming a mammalian cell with a gene or a construct comprising said gene encoding a CMP-SAT, a fragment or a variant thereof, at above endogenous levels; and
(b) transforming the cell with a gene or with a construct comprising said gene encoding at least a sialylated glycoprotein of interest.

Further, in the above method, the sialylated glycoprotein of interest may be produced at above endogenous levels. The method may further comprise a step (c) comprising the isolation of the glycoprotein(s) of interest. The isolated glycoprotein(s) may be formulated in the form of a pharmaceutical or therapeutic composition.

In a further related aspect, the invention comprises a method for producing a sialylated glycoprotein in a mammalian cell of interest, said method comprising the steps of:
(a) determining the carbohydrate substrates in said cell;
(b) transforming said cell with proteins to produce necessary precursor substrates; and
(c) constructing a processing pathway in said cell to produce a sialylated glycoprotein.

Various CMP-SAT proteins are known. In addition polynucleotide sequences encoding the CMP-SAT proteins used according to the methods of the invention are known, or are identified using bioinformatics searches. These sequences may be used in the present invention.

Suitable cells of interest include mammalian cells and cell lines. Human cells and cell lines are also included in the in the cells of interest and may be utilised. In particular, cells of interest include mammalian cells that are useful in the production of therapeutic glycoproteins. These cells may be in an unmodified state or may have been previously modified to express a therapeutic glycoprotein. Chinese hamster ovary cells have been found to be particularly useful in the production of glycoproteins for therapeutic use. It is envisaged that the use of the techniques of the present invention in combination with existing techniques will provide glycoproteins with above levels of sialylation above endogenous levels.

The methods and systems of the present invention may be used for a wide range of glycoproteins, which may be of therapeutic benefit. In particular the invention is useful where it the sialylation of the glycoprotein is desirable to prolong the circulatory time of the protein in the bloodstream. By way of example, suitable glycoproteins include interferon-gamma (IFN-γ).

The method and systems of the present invention, provide a further genetic manipulation process useable with existing techniques to modify a target cell to produce a ‘human’ glycoprotein. The glycoproteins produced by cells with the modification provided by the method and systems of the present invention are advantageous as they have increased sialylation which it is envisaged will lead to an increased circulatory time in the blood.

### DESCRIPTION OF THE FIGURES

FIG. 1. Primer design for real time PCR. To detect total CMP-SAT expression, primer set T was designed internal to the CMP-SAT open reading frame (ORF) where 5′-primer was 5′-TGATAAGTGTTGGACTTTTAGC-3′ (SEQ ID NO:1) and 3′-primer was 5′-CTTCAGTTGATAGGTAACCTGG-3′ (SEQ ID NO:2). To detect recombinantly expressed CMP-SAT, primer set R was designed such that the 5′-primer was within the CMP-SAT ORF and the 3′-primer flanked the ORF and the pCMV-Tag plasmid sequence, as shown in the figure. 5′-primer was 5′-CTGCAGCCATTGTTCTTTCTAC-3′ (SEQ ID NO:3) and 3′-primer was 5′-GTATCGATAAGCTTTCACACACC-3′ (SEQ ID NO:4).

FIG. 2. Protein PSI-Blast results of the PCR product obtained. The DNA sequence obtained was translated and blasted using PSI-Blast. A point mutation of valine to methionine was observed at amino acid 103.

FIG. 3. FACS analysis of transiently transfected cells. Cells were transiently transfected with negative control pcDNA3.1 (+) (A), actual plasmid pCMV-FLAG®-SAT (B), and positive control pCMV-FLAG®-Luc (C). A marker region M1, was used to arbitrarily define 1% of the cell population with higher fluorescence in the negative control. This same maker region defined 16.5% and 6.4% of the cell population with higher fluorescence for the actual plasmid and the positive control respectively. There was thus expression of the FLAG-CMP-SAT and FLAG-Luciferase in the respective samples.

FIG. 4. Real time PCR analysis of stable CMP-SAT clones versus untransfected CHO IFN-γ. When total CMP-SAT expression was compared (A), the expression in the positive clones was a fold higher than the untransfected CHO IFN-γ and the null cell line, IB.8. When recombinant CMP-SAT expression was compared (B), expression in the positive clones was distinctly higher than the CHO IFN-γ and IB.8, and the latter samples had threshold cycles closer to the negative control, where reaching a threshold cycle is caused by fluorescence due to primer-dimer formation. Expression levels could be compared this way since an equal amount of starting template was used in each case. Threshold cycle is defined as the cycle when a given sample crosses the Relative Fluorescence Unit, RFU value of 20,000. W represents a negative control run when water is used as the template.

FIG. 5. Sialic acid analysis using a modified thiobarbituric acid assay (Hammond and Papermaster, 1976). The average sialic acid content of the cell lines in moles sialic acid per mole IFNγ were: Untransfected CHO IFN-γ-2.61±0.07, IC.17-2.86±0.16, IC.30-2.83±0.17, IC.37-3.03±0.21, IC.38-2.85±0.21, IB.8-2.30±0.28. Using a 2-tailed Student's T test, average sialic acid content readings of each stable cell line was compared with values obtained from untransfected CHO IFN-γ. The p values obtained were less than 0.05 and measurements were considered statistically significant. The percentage values represent the percentage increase in average sialic acid content over the untransfected CHO IFN-γ.

FIG. 6. Glycan site occupancy data using MEKC. There were no distinct differences in the site occupancy of both the overexpressing CMP-SAT cell lines and the null cell line compared with the untransfected CHO IFN-γ. O-site glycosylated IFNγ could not be detected in the IFNγ obtained from some of the cell lines. The standard deviation was obtained from duplicate runs of each sample.

FIG. 7. Simplified diagram of the sialylation process. CMP-sialic acid is transported from the cytosol to the Golgi via the CMP-sialic acid transporter. This occurs through an antiport mechanism where the entry of CMP-sialic acid into the Golgi lumen is coupled to an equimolar exit of CMP from the lumen (Hirschberg et al., 1998). The sialyltransferase then utilizes CMP-sialic acid as a co-substrate for transfer of sialic acid onto an incoming polypeptide chain with a β1,4-galactose acceptor site.

FIG. 8. Real-time PCR primers used to detect total and recombinant CMP-sialic acid transporter expression. To detect total CMP-sialic acid transporter expression, primer set T was designed internal to the CMP-sialic acid transporter open reading frame (ORF) where forward primer was 5′-TGATAAGTGTTGGACTTTTAGC-3′ (SEQ ID NO:8) and reverse primer was 5′-CTTCAGTTGATAGGTAACCTGG-3′ (SEQ ID NO:9). To detect recombinant CMP-sialic acid transporter, primer set R was designed such that the forward primer was part of the pcDNA3.1 (+) vector sequence, 5′-CTAGCGCCACCATGGCTCAGG-3′ (SEQ ID NO:10) and the reverse primer was within the CMP-sialic acid transporter ORF, 5′-CTTCTGTGACACACACGGCTGTG-3′ (SEQ ID NO:11).

FIG. 9(A, B). FACS analysis of surface glycoproteins from CHO-K1 and Lec2 cells using WGA-FITC (A) and PNA-FITC (B). Since Lec2 cells are unable to sialylate its glycoproteins as a result of a defect in the CMP-sialic acid transport, the surface glycoproteins bind less to WGA-FITC and more with PNA-FITC as compared to surface glycoproteins from CHO-K1 which are sialylated.

FIG. 10(A,B). FACS analysis of Lec2 cells transiently transfected with pcDNA-SAT (Lec2-SAT) using WGA-FITC (A) and PNA-FITC (B). An arbitrary M1 gating was set to 1% of the Lec2 cells stained with WGA-FITC of higher fluorescence (to the right of the plot) (A). 40.9% of the Lec2 cell population responded to WGA-FITC binding with respect to the M1 gating. The same M1 gating was set to 5% of Lec2 cells stained with PNA-FITC of lower fluorescence (to the left of plot) (B). 37.3% of the Lec2 cell population shifted to the left and had less binding with PNA-FITC based on the M1 gating. This experiment was repeated and similar results were obtained.

FIG. 11. Comparison of total CMP-sialic acid transporter transcript in selected clones and negative controls, untransfected parent CHO IFN-γ and null vector cell line. The fold increase in CMP-sialic acid transporter transcript with respect to untransfected parent CHO IFN-γ was 17.0±3.3, 20.1±0.9, 5.6±0.6 and 2.2±0.1 for clones 9, 15, 21 and 26 respectively. For each sample, cell pellets were harvested twice to generate cDNA samples for real-time PCR analysis. In each real-time PCR run, each sample was run in duplicate.

FIG. 12. Comparison of recombinant CMP-sialic acid transporter (CMP-SAT) transcript in selected clones and negative controls, untransfected parent CHO IFN-γ and null vector cell line. Clones 9 (♦) and 15 (▪), which had higher fold increase in total CMP-SAT transcript, show corresponding higher levels of recombinant CMP-SAT transcript, as indicated by the lower threshold cycles. A similar trend was observed in clones 21 (▴) and 26 (X), with a lower fold increase in total CMP-SAT transcript. The negative controls are represented by untransfected parent CHO IFN-γ () and null vector cell line ( [symbol] ). For each sample, cell pellets were harvested twice to generate cDNA samples for real-time PCR analysis. In each real-time PCR run, each sample was run in duplicate. This diagram shows a representative run from the repeat analyses.

FIG. 13. Western blot analysis of adherent stable clones over expressing CMP-sialic acid transporter when compared to negative controls, untransfected parent CHO IFN-γ and null vector cell line. The CMP-sialic acid transporter was identified as an approximately 30 kDa band based on previous references (Berninsone et al., 1997; Eckhardt & Gerardy-Schahn, 1997; Ishida et al., 1998).

FIG. 14. Recombinant IFN-γ sialic acid content of adherent stable clones over expressing CMP-sialic acid transporter when compared to negative controls, untransfected parent CHO IFN-γ and null vector cell line (n=2). IFN-γ sialic acid content measurements from clones 9, 15 and 26 were statistically different from the untransfected parent CHO IFN-γ according to the Student's t-Test (p<0.05). The thiobarbituric acid assay, which was used to measure IFNγ sialic acid content, was carried out twice, where each sample was performed in duplicate.

FIG. 15. Recombinant IFN-γ site occupancy of adherent stable clones over expressing CMP-sialic acid transporter and negative controls, untransfected parent CHO IFN-γ and null vector cell line. The bars represent proportion of 2-N ( [symbol] ), 1-N (▪) and 0-N (□) glycan site occupied IFN-γ. The percentages represent average values obtained from 2 to 3 micellar electrokinetic capillary chromatography (MEKC) runs.

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

The methods of the present invention permit manipulation of glycoprotein production in cells of interest by enhancing the production CMP-SAT and/or the sialylation of glycoproteins.

By “cells of interest” is intended, for example, 1) any cells in which the endogenous CMP-SA levels are not sufficient for the production of a desired level of sialylated glycoprotein in that cell, 2) where it is desired to improve the rate of introduction of CMP-SA into the Golgi, 3) where it is desired to improve or enhance the amount or activity of CMP-SAT, or 4) where it is desired to improve or maximize the production of sialylated glycoproteins. The cell of interest can be any mammalian cell. For example, CHO cells. Human cells and cell lines are also included in the cells of interest and may be utilized according to the methods of the present invention. For example, they may be used to manipulate sialylated glycoproteins in human cells and/or cell lines, such as, for example, kidney, liver, and the like. By “desired level” is intended that the quantity of a biochemical comprised by the cell of interest is altered subsequent to subjecting the cell to the methods of the invention. In this manner, the invention comprises manipulating levels of CMP-SAT and/or sialylated glycoprotein(s) in the cell of interest. In a preferred embodiment of the invention, manipulating levels of CMP-SAT and/or sialylated glycoprotein comprise overexpression of CMP-SAT and/or sialylated glycoprotein(s), that is, increasing the levels of CMP-SAT and/or sialylated glycoprotein(s) to above endogenous levels.

For purposes of the present invention, by “enhancing expression” is intended to mean that the translated product of a nucleic acid encoding a desired CMP-SAT protein and/or the translated product of a nucleic acid encoding a desired glycoprotein is higher than the endogenous level of that protein(s) in the host cell(s) in which the nucleic acid(s) is expressed.

By “endogenous” is intended to mean the type and/or quantity of a biological function or a biochemical composition that is present in a naturally occurring or recombinant cell prior to manipulation of that cell according to the methods of the invention.

By “heterologous” is intended to mean the type and/or quantity of a biological function or a biochemical composition that is not present in a naturally occurring or recombinant cell prior to manipulation of that cell by the methods of the invention.

For purposes the present invention, by “a heterologous glycopolypeptide or glycoprotein” is meant as a glycopolypeptide or glycoprotein expressed (i.e. synthesized) in a cell species of interest that is different from the cell species in which the glycopolypeptide or glycoprotein is normally expressed (i.e. expressed in nature).

Methods for determining endogenous and heterologous functions and compositions relevant to the invention are provided herein; and otherwise encompass those methods known in the art.

The prior art literature showed that several limiting factors influence the efficient sialylation of glycoproteins in the cells. The present inventors propose that a limiting amount of CMP-sialic acid substrate in the Golgi available for sialylation is caused by a limitation in CMP-sialic acid transport into the Golgi via the CMP-sialic acid transporter (CMP-SAT). Accordingly, the present inventors overexpress the CMP-SAT to alleviate this limitation. This resulted in an increase in sialylation of glycoproteins produced in the cells, including the recombinant protein of interest.

The CMP-SAT(s) belong to the family of nucleotide sugar transporters whose structures and transport mechanisms are largely similar (reviewed in Berninsone & Hirschberg, 2000; Hirschberg et al., 1998; Hirschberg & Snider, 1987; Kawakita et al., 1998). The hamster CMP-sialic acid transporter cDNA was previously isolated through complementation cloning of Lec2 (Eckhardt & Gerardy-Schahn, 1997), a CHO glycosylation mutant cell line that had a defect in the CMP-sialic acid transporter (Deutscher et al., 1984). The human (Ishida et al., 1998) and murine (Berninsone et al., 1997) homologs of the CMP-sialic acid transporter have been demonstrated to have functional activity and the similarly cloned hamster homolog was expected to have similar functionality. The membrane topology of this transmembrane protein has also been studied extensively (Eckhardt et al., 1999).

The present invention solves the problems addressed in the prior art and provides an efficient method and system for producing sialylated glycoproteins, which mitigates the forgoing problems. In particular, the present invention provides for a method for the preparation of sialylated glycoprotein(s) of interest in a cell, isolated cell, or cell line comprising enhancing the expression of a CMP-SAT, a fragment or a variant thereof, at above endogenous level. The present inventors have surprisingly found that enhancing the production of CMP-SAT at above the endogenous level allowed an efficient sialilyzation of glycoproteins. In particular, the method of the invention is an efficient method for maximizing the sialic content of glycoproteins on interest. The methods and systems of the invention are useful for producing complex sialylated glycoproteins in mammalian cells of interest including, but not limited to, CHO cells.

According to a first aspect, the present invention provides at least a mammalian cell producing a CMP-SAT, a fragment or a variant thereof, at above endogenous levels. The mammalian cell may be any suitable mammalian cell for the purposes of the present invention, for example a human or CHO cell. The invention also provides a cell line, for example in the form of a cell culture comprising the cell producing a CMP-SAT and/or sialylated glycoproteins of interest at above endogenous levels. Suitable cells of interest include mammalian cells and cell lines. Human cells and cell lines are also included in the in the cells of interest and may be utilised. In particular, cells of interest include mammalian cells that are useful in the production of therapeutic glycoproteins. These cells may be in an unmodified state or may have been previously modified to express a therapeutic glycoprotein. Chinese hamster ovary cells have been found to be particularly useful in the production of glycoproteins for therapeutic use. It is envisaged that the use of the techniques of the present invention in combination with existing techniques will provide glycoproteins with above levels of sialylation above endogenous levels.

According to one aspect of the invention, the increase in sialylation is achieved by expressing a CMP-SAT protein, or a fragment or variant thereof, in the cell of interest. Accordingly, the invention provides at least one mammalian cell, wherein the cell produces at least one sialylated glycoprotein at above endogenous levels. In particular, the mammalian cell produces a CMP-SAT, a fragment or a variant thereof, at above endogenous levels, and at least one sialylated glycoprotein at above endogenous levels. The at least one glycoprotein may be heterologous. The glycoprotein may be mammalian, for example human or CHO glycoprotein. However, any other glycoprotein useful for the purposes of the present invention may be produced. In particular, the cell of the invention may be used for the production of a complex or mixture of sialylated glycoproteins of interest.

More in particular, the model system that was used in the experimental part of the present invention was CHO IFN-γ, a Chinese hamster ovary cell line producing human IFN-γ. However, IFN-γ from other mammalian sources, may also be used. Further, other forms of IFN, for example IFN-alpha or -beta may also be used. IFN-γ is a secretory glycoprotein with antiviral, antiproliferative and immunomodulatory activities (Farrer & Schreiber, 1993). There are 2 potential N-glycosylation sites at Asn-25 and Asn-97 which are variably occupied. When occupied, the oligosaccharides are predominantly biantennary (Gu et al., 1997; Hooker et al., 1995). Accordingly, the glycoprotein of interest may be any IFN-γ, a fragment or a variant thereof. With the term “a fragment or a variant thereof”, it is intended a fragment and/or a variant expressing the biological activity of the IFN-γ.

Accordingly, the mammalian cell or cell line of the invention may be transformed with a gene encoding a CMP-SAT and/or a heterologous protein or with a construct comprising that gene. For example, the cell is transformed with a gene encoding at least an IFN-γ, a fragment or a variant thereof. Therefore, the invention comprises the genetic engineering of cells with a CMP-SAT gene so that the cell expresses the CMP-SAT protein at a level above the endogenous level. The increase in CMP-SAT expression allows for the increased transport of the CMP-sialic acid (CMP-SA) into the Golgi apparatus in order to obtain sialylation of glycoprotein(s) at above endogenous levels. Accordingly, it is provided at least a mammalian cell, wherein the cell is transformed with a gene encoding CMP-SAT or a construct comprising that gene. In particular, the mammalian cell is transformed with a mammalian CMP-SAT gene. For example, with a human or CHO CMP-SAT gene.

Expression cloning of multiple transcripts (for example, transcripts encoding CMP-SAT and/or glycoproteins of interests) in a single cell line using techniques known in the art may be required to bring about the desired sialylation reactions and/or to optimize these reactions. Alternatively, co-infection of cells with multiple viruses using techniques known in the art can also be used to simultaneously produce multiple recombinant transcripts. In addition, plasmids that incorporate multiple foreign genes including some under the control of the promoter or early promoter are commercially, publicly, or otherwise available for the purposes of the invention, and can be used to create suitable constructs. The present invention encompasses using any of these techniques. The invention also encompasses using the above mentioned types of vectors to enable expression of desired CMP-SAT in cells prior to production of a heterologous glycoprotein of interest. Alternatively, genes for CMP-SAT and/or the glycoprotein(s) of interest may be incorporated directly into the host cell genome using vectors known in the art. In addition, a sequential transformation strategy may routinely be developed for producing stable transformants that constitutively express one or more different heterologous genes simultaneously. In particular, the mammalian cell or cell line of the invention may be a CHO cell, isolated cell or cell line and the cell produces CMP-SAT, a fragment or a variant thereof, at above endogenous levels, wherein the cell produces at least one sialylated glycoprotein at above endogenous levels. The sialylated glycoprotein may be at least IFN-γ, a fragment or a variant thereof. The mammalian cell of the invention may be an isolated cell line.

According to another aspect, the present invention provides a kit for the expression of at least one sialylated glycoprotein comprising the mammalian cell or cell line of the invention.

According to another aspect, methods and systems for producing glycoproteins having sialylated oligosaccharides are provided. Accordingly, the invention provides a method for the preparation of sialylated glycoprotein(s) in a cell or cell line comprising enhancing the expression of a CMP-SAT, a fragment or a variant thereof, at above endogenous levels. According to a further aspect, the sialylated glycoprotein(s) is also expressed at above endogenous levels. More in particular, the overexpression of CMP-SAT brings about a maximum sialylation of glycoprotein(s). In particular, the mammalian cell or cell line may be CHO or human cell line. The sialylated glycoprotein(s) may be a heterologous mammalian glycoprotein. For example, the sialylated glycoprotein(s) is any IFN-γ, a fragment or a variant thereof.

More in particular, in the present invention, the authors report the overexpression (that is, expression above endogenous level) of the hamster CMP-SAT in CHO IFN-γ. The present inventors herein proved that overexpression of CMP-SAT lead to an improvement in the sialylation of recombinant IFN-γ. As this glycosylation engineering approach in CHO cells is not known to be reported elsewhere, it represents a novel approach to improve sialylation during recombinant glycoprotein production.

According to another aspect, the invention provides a method for producing at least a sialylated glycoprotein in a mammalian cell or cell line comprising the steps of:
(a) transforming a mammalian cell with a gene or a construct comprising said gene encoding a CMP-SAT, a fragment or a variant thereof, at above endogenous levels; and
(b) transforming the cell with a gene or with a construct comprising said gene encoding at least a sialylated glycoprotein of interest.

Further, in the above method, the sialylated glycoprotein of interest may be produced at above endogenous levels. The method may further comprise a step (c) comprising the isolation of the glycoprotein(s) of interest. The isolated glycoprotein(s) may be formulated in the form of a pharmaceutical or therapeutic composition.

In a further related aspect, the invention comprises a method for producing a sialylated glycoprotein in a mammalian cell of interest, said method comprising the steps of:
(a) determining the carbohydrate substrates in said cell;
(b) transforming said cell with proteins to produce necessary precursor substrates; and
(c) constructing a processing pathway in said cell to produce a sialylated glycoprotein.

The cell may be an isolated cell or cell line.

Various CMP-SAT proteins are known. In addition polynucleotide sequences encoding the CMP-SAT proteins used according to the methods of the invention are known, or are identified using bioinformatics searches. These sequences may be used in the present invention.

The methods and systems of the present invention may be used for a wide range of glycoproteins, which may be of therapeutic benefit. In particular the invention is useful where in the sialylation of the glycoprotein is desirable to prolong the circulatory time of the protein in the bloodstream. By way of example, suitable glycoproteins include interferon-gamma (IFN-γ).

In particular, the present inventors have established a novel strategy for improvement of recombinant protein sialylation. The inventors have herein confirmed that overexpression of CMP-SAT lead to increased sialylation in CHO cells, through the use of their model cell line, CHO IFN-γ. The increase of 4 to 16% IFN-γ sialylation by the clones overexpressing CMP-SAT was comparable to existing glycosylation engineering approaches. In fact, it is also demonstrated that overexpression of CMP-SAT alone is sufficient to bring about maximal sialylation of IFN-γ in some of the clones. More significantly, for a given level of IFN-γ site occupancy and branching, maximal sialylation of the recombinant protein product has been obtained through this strategy. The increase in CMP-sialic acid substrate availability in the Golgi through CMP-sialic acid transporter overexpression was sufficient to fully sialylate the recombinant IFN-γ in some of the chosen single cell clones. This is the ideal case in recombinant protein production, since fully sialylated glycoproteins have prolonged in vivo circulatory half-life.

The effectiveness of CMP-SAT overexpression strategy depends on the cell-type variations in glycosylation machinery as well as cellular demand for sialic acid. There is a variation in endogenous CMP-SAT expression in different cell lines and this strategy should therefore prove more effective in cell lines with low amounts of CMP-SAT. Alternatively, if the supply of CMP-sialic acid is artificially increased through exogenous feeding, for example through N-acetylmannosamine (ManNAc) feeding, the overexpressed CMP-sialic acid transporter serves to transport the increased CMP-sialic acid substrate into the Golgi with possible improvement in siaylation through this combined approach. If maximal sialylation had been achieved in IFN-γ through CMP-SAT overexpression alone, additional ManNAc feeding may result in no further improvement of sialylation.

The cellular demand for sialic acid depends on the number of β1,4-galactose acceptor sites in the Golgi lumen (Baker et al., 2001). This in turn depends on the type of recombinant glycoprotein produced and hence the amount of sialylation that is involved, as well as the rate of its production or specific productivity. It should be noted that the parent CHO IFN-γ produces IFN-γ with relatively high levels of sialylation (Table 2). This could be partly attributed to CHO IFN-γ being a low yielding cell line, with a small number of recombinant protein molecules passing through the Golgi per unit time, resulting in low sialylation demand.

It is submitted that the strategy of CMP-SAT overexpression is generally a useful strategy to adopt for sialylation improvement, especially if the cell line has high recombinant protein productivity or lower basal sialic acid content as compared to the model glycoprotein that is used. In addition, the results demonstrate the possibility of considering CMP-SAT together with glycosyltransferases for genetic manipulation of the glycosylation pathway, as well as nucleotide sugar feeding in a multi-prong approach to improve glycosylation. For example, N-acetylmannosamine feeding coupled with CMP-SAT would allow increased transport of the increased CMP-sialic acid substrate into the Golgi with possible enhanced improvement in siaylation. Such multi-prong approaches can be applied to a wide variety of recombinant glycoproteins and also allow the achieving of maximum and consistent sialylation in glycoprotein production using mammalian cells.

The invention encompasses expressing heterologous proteins in the cells of the invention and/or according to the methods of the invention for any purpose benefiting from such expression. Such a purpose includes, but is not limited to, increasing the in vivo circulatory half life of a protein; producing a desired quantity of the protein; increasing the biological function of the protein including, but not limited to, enzyme activity, binding capacity, antigenicity, therapeutic property, capacity as a vaccine or a diagnostic tool, and the like. Such proteins may be naturally occurring chemically synthesized or recombinant proteins. Examples of proteins that benefit from the heterologous expression of the invention include, but are not limited to, transferrin, plasminogen, thyrotropin, tissue plasminogen activator, erythropoietin, interleukins, and interferons. Other examples of such proteins include, but are not limited to, those described in International patent application publication number WO 98/06835, the contents of which are herein incorporated by reference. In one embodiment, proteins that benefit from the heterologous expression of the invention are mammalian proteins. In this aspect, mammals include but are not limited to, Chinese hamsters, cats, dogs, rats, mice, cows, pigs, non-human primates, humans, and the like.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Sambrook and Russel, Molecular Cloning: A Laboratory Manual, Cold Springs Harbor Laboratory, New York (2001).

Those skilled in the art will be familiar with various techniques useable to introduce a gene for a CMP-SAT protein into a mammalian cell of interest. Specifically the following establishes an acceptable approach which we have found to be effective in genetically engineering the cell of interest to express CMP-SAT at an above endogenous level and thereby to achieve a sialylation of glycoproteins at an above endogenous level. As those skilled in the art will appreciate, this genetic engineering and expression may be achieved using methods that differ in their detail from those described below. By way of example, different vectors and host cells may be used.

### Example 1

### Materials and Methods

### Mammalian Cell Lines and Media

A CHO cell line expressing human IFN-γ (Scahill et al., 1983) was used for the cloning work. This cell line, referred to as CHO IFN-γ was grown in Dulbecco's Modified Eagle Medium (DMEM) (Invitrogen, Grand Island, N.Y.) supplemented with 10% (v/v) fetal bovine serum (HyClone, Logan, Utah) and 0.25 μM methothrexate. The methothrexate was added to maintain the selection pressure in this DHFR cell line, but this was removed during the initial Geneticin selection period for stable cell lines, which is mentioned later. Cells were grown as monolayers in stationary T-flasks and incubated at 37° C. under a 5% CO₂ atmosphere. Cells were detached from T-flasks by adding 0.05% (v/v) trypsin/EDTA solution (Sigma, St. Louis, Mo.) during regular sub-culturing.

Full Length cDNA Synthesis from CHO-K1

Total RNA was prepared from CHO-K1 by the SV Total RNA Isolation System (Promega, Madison, Wis.) according to manufacturer's instructions. All reverse transcription reagents were from Promega. Full length cDNA was synthesized using Moloney Murine Leukaemia Virus Reverse transcriptase (M-MLV RT) for 1 hour at 42° C. in a reaction mix containing 5×M-MLV reaction buffer, 10 mM of each dNTP and 25 units of recombinant RNAsin® ribonuclease inhibitor.

### Polymerase Chain Reaction (PCR) Amplification of CMP-SAT

The cDNA prepared from CHO-K1 total RNA was used as a template to amplify the coding region of the CMP-SAT cDNA, based on primers designed from the previously cloned hamster CMP-SAT (Eckhardt and Schahn, 1997). BamHI and HindIII restriction sites were introduced upstream and downstream of the coding region for subsequent subcloning. The 5′-PCR primer used was 5′-ATAGGATCCTGCTCAGGCGAGAGA-3′ (SEQ ID NO:5) and the 3′-PCR primer used was 5′-GACAAGCTTTCACACACCAATGAC-3′ (SEQ ID NO:6), where the introduced restriction sites are underlined, and the incorporated coding regions of the CMP-SAT are in bold. All PCR reagents were from Promega. The reaction mix contained 2 μl of DNA template, 1×Pfu buffer, 250 μM of each dNTP, 1 μM of each primer and a Taq-Pfu polymerase mix (approximately 5 U). PCR conditions were: 94° C. for 6 minutes, followed by 35 cycles of 94° C. for 1 minute, 50° C. for 1 minute, and 72° C. for 1 minute, and a final extension at 72° C. for 8 minutes.

### Construction of CMP-SAT Expression Vector

The PCR product was first subcloned into pCR®-TOPO® (Invitrogen, Grand Island, N.Y.) for sequencing, where it was compared with the previously cloned hamster CMP-SAT sequence for any mutations. The expression vector chosen was the pCMV-Tag vector (Strategene, La Jolla, Calif.), containing a FLAG® epitope (DYKDDDDK) (SEQ ID NO:7) at the N-terminus. The verified PCR product was subcloned into pCMV-Tag and sequenced again. Since a fusion protein was to be produced, it was important to ensure the FLAG® sequence was in frame with the coding region of the CMP-SAT for correct expression. The final plasmid pCMV-FLAG-SAT, was purified using the Maxi Plasmid Purification Kit (Qiagen, Hilden, Germany) and its concentration quantified for transfection into CHO IFN-γ.

Transient and Stable Transfection of DNA into CHO-IFNγ

Before the actual transfection was carried out, the CHO IFN-γ was titered against varying concentrations of Geneticin (Sigma, St. Louis, Mo.) between 0.1 to 1.0 μg/ml to determine the minimum concentration of Geneticin required to kill the untransfected CHO IFN-γ. The transfection was carried out using Fugene 6 transfection reagent (Roche, Basel, Switzerland). Cells were grown overnight in 6-well plates with 0.5 million cells per well and transfected with approximately 1 μg of circular plasmid per well the next day. The Fugene-DNA complex was prepared according to manufacturer's instructions in a 6:1 Fugene 6 transfection reagent (μl) to DNA (μg) ratio.

For transient transfections, cells were grown for 48 hours before they were harvested for FACS analysis. For generation of stable cell lines, the cells were grown for 48 hours before the media was changed to selection media containing 700 μg/ml of Geneticin, as determined in earlier titering experiments. The cells were maintained in the selection media for 3 weeks, where the untransfected cells in the selection media died within a week. After 3 weeks, Geneticin-resistant colonies were observed and these colonies were randomly picked and subsequently expanded to stable cell lines. These cells lines were maintained for 6 passages in selection media before Geneticin was removed.

### FACS Analysis

FACS analysis was carried out by labeling cells intracellularly with anti-FLAG® M1 mouse monoclonal antibody (Sigma, St. Louis, Mo.). CHO IFN-γ was transiently transfected with the following vectors: pcDNA3.1 (+) (Invitrogen, Grand Island, N.Y.), pCMV-FLAG®-Luc (Strategene, La Jolla, Calif.) and pCMV-FLAG®-SAT using Fugene 6 transfection reagent. pCMV-FLAG®-Luc is a positive control vector that results in expression of FLAG®-Luciferase protein. Approximately 1.5 million cells were used in each FACS preparation. The cells were washed in PBS and resuspended to obtain a single cell suspension. This suspension was fixed and permeabilised using a Fix & Perm® Cell Permeabilisation Kit (Caltag Laboratories, Burlingame, Calif.). They were then labeled with 1:870 dilution of anti-FLAG® M1 mouse monoclonal antibody for 15 minutes. Cells were subsequently washed in 1% (w/v) bovine serum albumin (BSA) (Sigma) in PBS (1% BSA/PBS) and incubated with 1:500 dilution of secondary anti-mouse IgG FITC (Dako, Copenhagan, Denmark) for 15 minutes in the dark. After a final wash in 1% BSA/PBS, cells were resuspended in fresh 1% BSA/PBS and analyzed using the FACSCalibur™ System (BD Biosciences, San Jose, Calif.). Results were analyzed using the accompanying software's analysis tools.

Total RNA Extraction and cDNA Synthesis from CHO IFN-γ Clones

Approximately 10 million cells were collected from stable cell lines and total RNA was extracted using TRIzol™ reagent (Invitrogen, Grand Island, N.Y.) as follows. Cells were resuspended in 1 ml of Trizol™ reagent and syringe-sheared 50 times with a 21 G needle. Cells were incubated at room temperature for approximately 10 minutes. 200 μl of chloroform was added and mixed vigorously for 30 seconds. The samples were then microfuged at 14,000 rpm for 15 minutes at 4° C. The upper aqueous layer was transferred to a new RNAse-free tube and an equal volume of isopropanol was added. The tubes were then incubated at −20° C. for 2 hours or more. Tubes were subsequently thawed on ice and microfuged at 14,000 rpm for 15 minutes at 4° C. A visible RNA pellet was seen at the bottom of the tube. The supernatant was aspirated and pellet was washed with 0.5 ml of 75% (w/v) ethanol. The pellet was then microfuged at 14,000 rpm for 2 minutes at 4° C. and the ethanol removed. The RNA pellet was air-dried and dissolved in 35 μl of DEPC water. A sample was taken for RNA quantification using the GeneQuant™ Pro RNA/DNA Calculator (Amersham Biosciences, Piscataway, N.J.). RNA quality was assessed using the absorbance ratio of 260 nm to 280 m, where a ratio of 1.9 and above was considered an indicator of RNA with sufficient purity.

Based on the RNA concentrations, 10 μg of RNA was used to synthesize first-strand cDNA. Reverse transcription was carried out with 400 U of Improm-II reverse transcriptase and 0.5 μg of Random Primers (Promega, Madison, Wis.) at 42° C. for 60 minutes according to manufacturer's instructions. The reaction was terminated at 70° C. for 5 minutes, and cDNA was used for subsequent real-time PCR analysis.

### Real-Time PCR

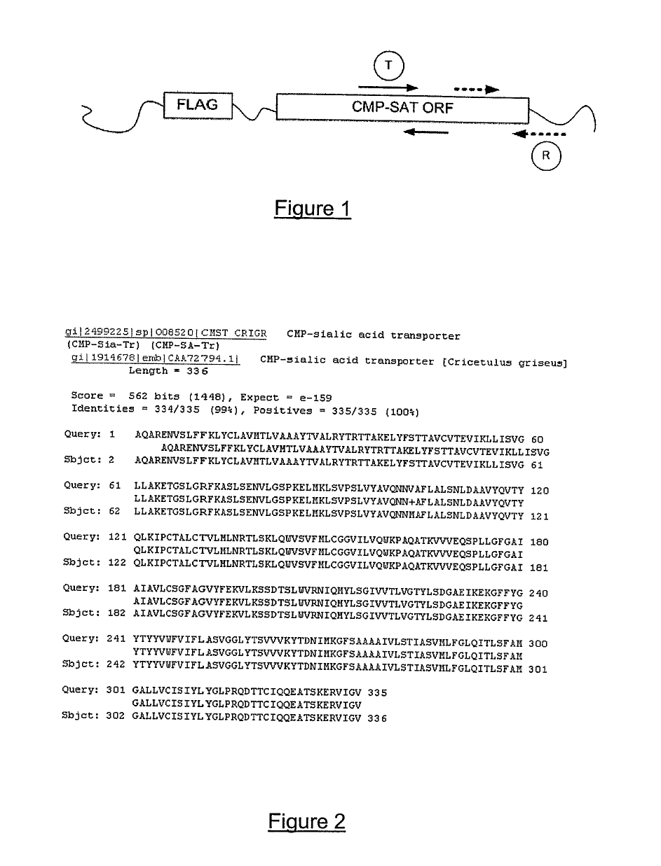

Real-time PCR was carried out using the iCycler iQ (Biorad, Hercules, Calif.) courtesy of Professor Heng-Phon Too, (Singapore-MIT Alliance, National University of Singapore). PCR conditions were: 95° C. for 3 minutes, followed by 40 cycles at 95° C. for 60 seconds, 55° C. for 30 seconds, and 72° C. for 60 seconds. Fluorescent detection was carried out during the annealing phase. The reaction buffer of 100 μl 1× XtensaMix-SG™ (BioWORKs) contained 2 mM MgCl₂, 10 pmol forward and reverse primers, 1.0 U DyNAzyme II (Finnzymes Oy, Espoo, Finland) and 5 μl of cDNA from CHO IFN-γ samples as prepared above. Samples in 40 μl aliquots were run in duplicate during each run. Primers for detection of total CMP-SAT and recombinantly expressed CMP-SAT were designed as shown in FIG. 1 (SEQ ID NOS:1-4).

### Sialylation Analysis of IFNγ

Cultures of stable clones and untransfected CHO IFN-γ were seeded at 2.5E6 cells in a T150 culture flask with 25 ml of media. The stable clones analyzed were IC.17, IC.30, IC.37, IC.38, and IB.8, as will be described below. The supernatant was harvested at t=92 h of the culture, which was the time of peak density of the cultures. The collected supernatant was pooled from 2 flasks and used for subsequent IFN-γ analysis. IFN-γ analysis was carried out using in-house optimized analysis procedures in Bioprocessing Technology Institute, based on previous work by Gu (MIT, PhD Thesis, 1997). In summary, the supernatant was immunopurified for IFN-γ. This purified IFN-γ was then quantified using reverse phase HPLC, where standards of known IFN-γ concentration had been run and compared with the actual samples. Total sialic acid was measured using a modified version of the thiobarbituric acid assay (AA) (Hammond and Papermaster, 1976). 4 to 5 μg of purified IFN-γ is used for each assay sample, where sialic acid is cleaved from IFN-γ using sialidase (0.0025 U each) (Roche, Basel, Switzerland) treatment before the actual assay This is because the assay only measures free sialic acid. The TAA was repeated 3 times, where each sample was run in duplicate. A total of 6 to 8 measurements were used for comparison in the 2-tailed Student's T-test. In addition, site occupancy of the IFN-γ was also measured using micellar electrokinetic capillary chromatography (MEKC). Each sample run was carried out twice.

### Results

Establishment of CHO-IFN-γ Cell Lines with Overexpressed CMP-Sialic Acid Transporter

The PCR amplification of the full length CMP-SAT was carried out based on primers designed around the CMP-SAT cDNA as described earlier. The sequence obtained was almost 100% similar to the published sequence, except for a point mutation of valine to methionine at amino acid position 103 This was repeatedly detected during separate sequencing runs for different clones obtained during the sub-cloning procedure (FIG. 2).

Since the actual 3-D protein structure of CMP-SAT was not known, it was hard to predict the effects of this mutation on the secondary structure of the protein. This mutation was thus assumed to give negligible effects on the protein expression and the PCR product used for subsequent sub-cloning.

A FLAG®-CMP-SAT fusion protein was chosen for expression since the presence of FLAG® differentiated the recombinant CMP-SAT from the endogenous protein. Moreover, the FLAG® fusion protein facilitated protein detection work since the antibody against the actual protein was not available. In addition, it was shown that FLAG® did not affect the localization and functional activity of CMP-SAT (Eckhardt and Schahn, 1997), unlike other epitope tags like the hemmaglutinin tag (Berninsone et al., 1997). The final plasmid pCMV-FLAG®-SAT was purified to a concentration between 0.4 to 0.6 μg/ml.

Some control cell lines were produced together with the actual cell lines containing overexpressed CMP-SAT. CHO IFN-γ was transfected with the null vector, pCMV-Tag and 14 colonies were picked and subsequently expanded to generate cell lines. One null vector cell line, IB.8 was used for subsequent sialylation analysis. A set of untransfected CHO IFN-γ cells were grown together with these cell lines to maintain passage history. 39 colonies were picked from transfected cells containing pCMV-FLAG®-SAT. 4 of these cell lines, IC.17, IC.30, IC.37 and IC.38 were chosen for subsequent sialylation analysis. The “selection basis” will be described in the next section.

### FACS Analysis of Overexpressed CMP-Sialic Acid Transporter in Transiently Transfected Cells

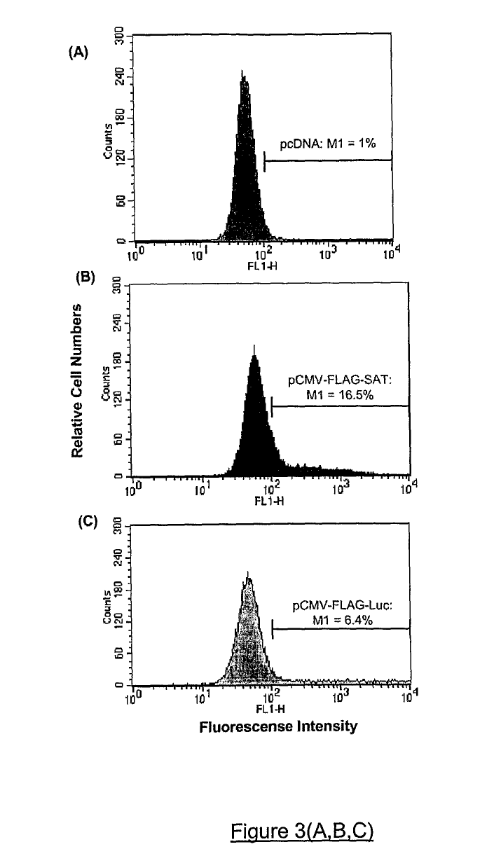

FACS analysis was carried out to detect FLAG®-CMP-SAT expression in transfected cells. As a negative control, cells transfected with pcDNA3.1 (+) was used. The null vector pCMV-Tag was not used since the FLAG® epitope would still be expressed and detected by FACS. In addition, it was found that negative control cells which had gone through the same process of transfection made a better control than untransfected cells. The results of the FACS analysis are shown in FIG. 3. The results obtained were as expected. To establish a basis of comparison, a marker region M1, was used to arbitrarily define 1% of the cell population with higher fluorescence in the negative control. This same maker region defined an increase to 16.5% of the cell population for the cells transfected with pCMV-FLAG®-SAT, indicating the expression of FLAG®-CMP-SAT. In addition, an increase to 6.4% of the cell population in the positive control indicated the expression of FLAG®-Luciferase.

With the above results from transient transfection experiments, it was expected that stable cell lines would result in a more significant shift of the cell population to contain higher fluorescence. As such, this FACS procedure could be used to screen the clones for relative expression of CMP-SAT. However, the population shift could not be demonstrated in the FACS analysis of the stable clones. Nevertheless, the comparison of marked populations in the various clones enabled random selection of “high”, “medium” and “low” expressers for further analysis with real-time PCR and sialylation analysis. This resulted in the 4 cell lines being selected for analysis as described in the earlier section.

### Real-Time PCR for Detection of Overexpressed CMP-Sialic Acid Transporter in Stable Cell Lines

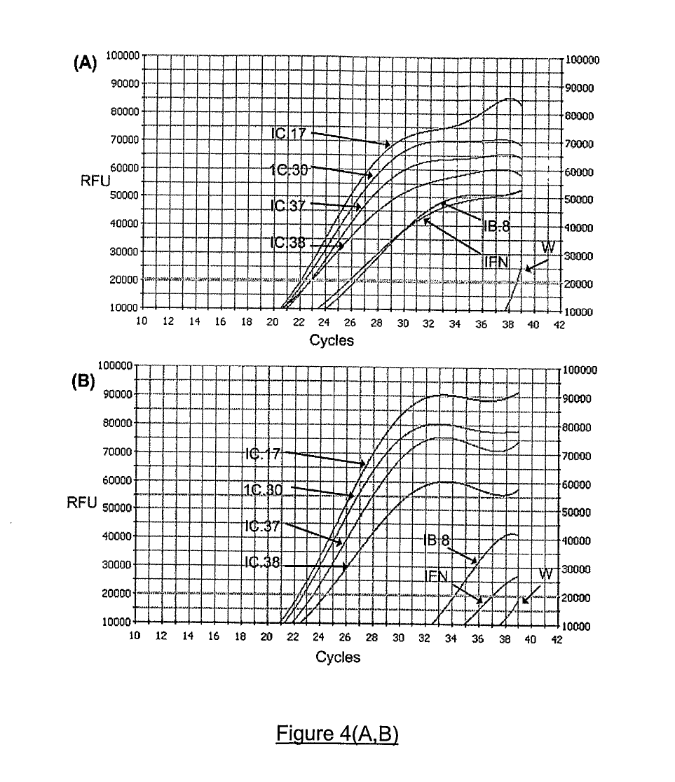

Real-time PCR is considered a sensitive method to detect low transcript levels (Bustin, 2000). It was thus considered suitable for comparing the expression of CMP-SAT in the overexpressing CHO IFN-γ clones versus the untransfected CHO IFN-γ. In addition, due to the specificity of the primers in amplifying selected gene regions, the 2 primer sets T and R (SEQ ID NOS:1-4) as described in the earlier section would allow comparison of total and recombinant expression of CMP-SAT. Results of the real-time PCR are found in FIG. 4.

The results obtained for the stable cell lines from real-time PCR were more conclusive compared to the FACS analysis. In this case, when the expression of CMP-SAT was compared amongst stable cell lines, a distinct difference could be seen. From (A) of FIG. 4, each of the 4 clones IC.17, IC.30, IC.37 and IC.38 had similar threshold cycles, which on average indicated a one fold higher expression of total CMP-SAT compared to the untransfected CHO IFN-γ. The results were even more apparent when recombinant CMP-SAT expression was measured (B). Each of the 4 clones showed expression, whereas the untransfected CHO IFN-γ and IB.8 had threshold cycles similar to the negative control performed using water as a template, where reaching a threshold cycle is caused by fluorescence due to primer-dimer formation. This confirmed recombinant CMP-SAT expression in the clones, which resulted in an overall increase in expression of total CMP-SAT in the positive clones.

### Sialylation Analysis of Recombinant IFN-γ in Stable Cell Lines

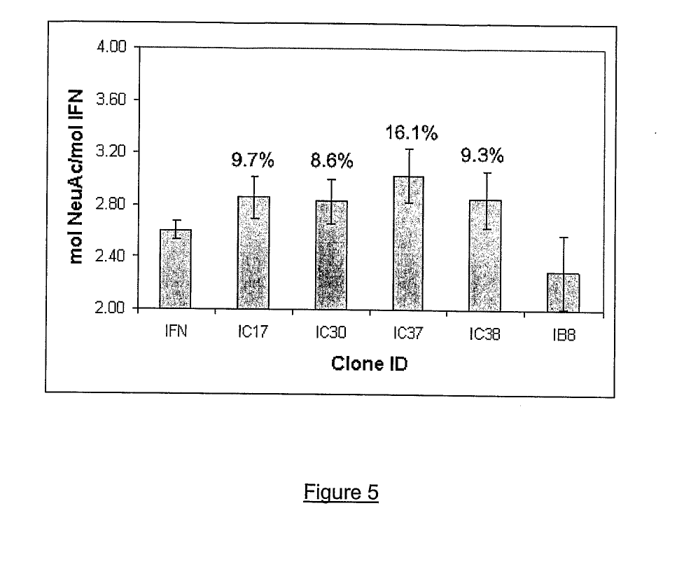

Overexpression of CMP-SAT was expected to improve the sialylation extent of IFN-γ produced by the CHO IFN-γ. An increase in transport ability of the CMP-sialic acid into the Golgi would lead to an increase in the CMP-sialic acid pool inside the Golgi for improved sialylation. This was found to be the case for the stable cell lines over-expressing CMP-SAT as described below. FIG. 5 shows the results obtained from the TAA assay that measured average sialic acid content of IFN-γ, where the amount of sialic acid was normalized to the amount of IFN-γ analyzed. As can be seen, there was an increase of between 8.6% to 16.1% in average IFN-γ sialic acid content of all the positive cell lines chosen, when compared with the control untransfected CHO IFN-γ (2.61±0.07 mol sialic acid per mol IFN-γ). Moreover, the average sialic acid content of the null cell line, IB.8 was lower than CHO IFN-γ, showing that the effects of sialic acid increase was due to the overexpression of the CMP-SAT, rather than clonal differences.

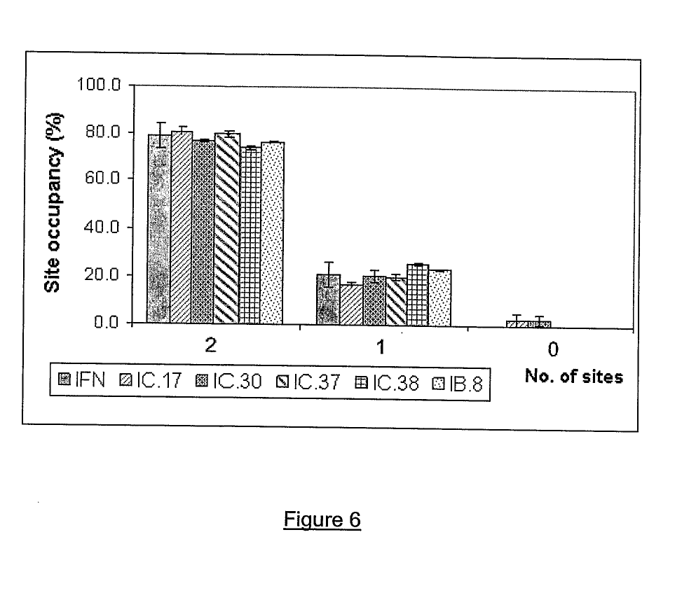

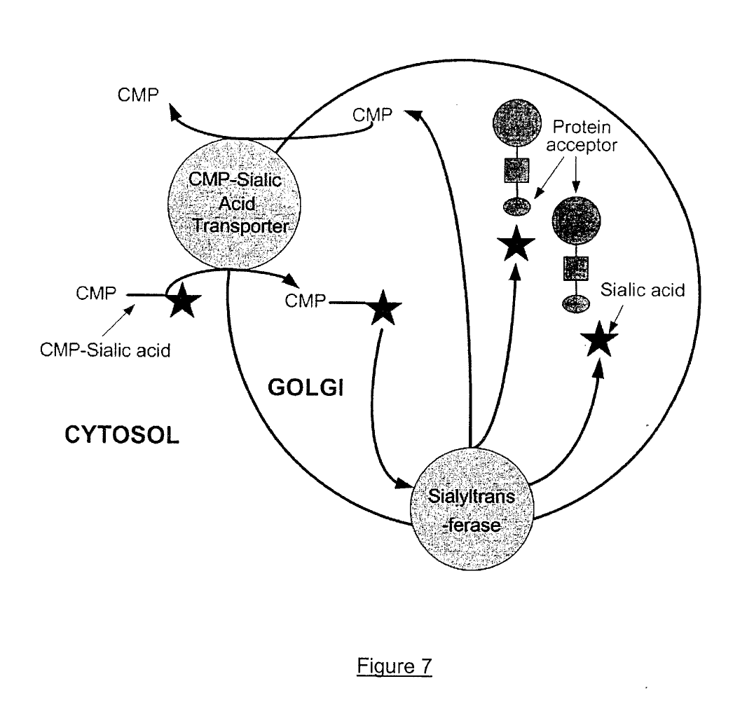

The site occupancy of the IFN-γ was measured and results are shown in FIG. 6. As expected, the overexpression of CMP-SAT did not significantly affect the site occupancy of IFN-γ, since the overexpression of CMP-SAT does not affect the transfer of the glycan to the protein, which is what influences site occupancy. However, the site occupancy data was used to normalize the average sialic acid content of IFN-γ against the number of available N-linked sites, to give a more accurate index of measurement known as site sialylation, as in equation (1). This normalized index enables us to directly consider the ability of the cell to sialylate an available site when manipulated by various conditions.

$$IFN γ site sialylation = \frac{IFN γ sialic acid content}{0.01 [ 2 ( \% 2 N ) + 1 ( \%1 N ) + 0 ( \%0 N ) ]} \qquad ( 1 )$$

where %2N, %1N and 0% N is the percentage of 2-sites, 1-site and 0-site glycosylated IFN-γ respectively.

Table 1 shows the IFN-γ site sialylation data. The maximum percentage increase in sialylation over the untransfected CHO IFN-γ was 16%, as analyzed on IFN-γ obtained from IC.37 cultures.

TABLE 1
IFN-γ SIALIC ACID CONTENT

| | Average sialic acid | | Site sialylation | |
|---|---|---|---|---|
| | content (mole sialic | Number of glycans | (molecules sialic | |
| Cell line | acid/mole IFN-γ) | per IFN-γ^{a} | acid/site) | % increase^{b} |
| IFN-γ^{c} | 2.61 ± 0.07 | 1.79 | 1.46 | |
| IC.17 | 2.86 ± 0.16 | 1.78 | 1.61 | 10.3 |
| IC.30 | 2.83 ± 0.17 | 1.74 | 1.63 | 11.6 |
| IC.37 | 3.03 ± 0..21 | 1.80 | 1.68 | 15.7 |
| IC.38 | 2.85 ± 0.21 | 1.74 | 1.64 | 12.6 |
| IB.8 | 2.30 ± 0.28 | 1.77 | 1.30 | (10.7) |

^{a}Number of glycans per IFN-γ was calculated based on site occupancy data. Its formula is the denominator of (1).
^{b}% increase was the percentage increase in site sialylation as compared with the untransfected CHO IFN-γ
^{c}IFN-γ represents the untransfected CHO IFN-γ.

This approach to overexpress the CMP-SAT to increase sialylation in recombinant glycoprotein therapeutics has not been reported elsewhere, to our best knowledge. Similar genetic engineering approaches to improve sialylation involve the overexpression of sialyltransferases. It has been reported the sialyltransferase engineering has led to sialylation increases of 23% in IFN-γ (Fukuta et al., 2000) and 30% in TNFR-IgG (Weikert et. al., 1999). The feeding of sialic acid precursor, N-acetylmannosamine achieved a 15% increase in sialylation in IFN-γ (Gu & Wang, 1998). Thus, the sialylation increase obtained through the overexpression of CMP-sialic acid transporter is comparable to past approaches. This approach is thus considered a novel means of improving sialylation in recombinant glycoprotein therapeutics.

### Example 2

### Materials and Methods

### CHO Cell Lines and Media

A CHO cell line expressing human IFN-γ referred to as CHO IFN-γ (Scahill et al., 1983) was used for the overexpression of CMP-sialic acid transporter. This cell line was created by co-transfecting genes for human interferon-gamma (IFN-γ) and dihydrofolate reductase (DHFR) in a DHFR deficient CHO cell line. The adherent cell line was maintained in Dulbecco's Modified Eagle Medium (DMEM) (Invitrogen, Grand Island, N.Y.) supplemented with 10% (v/v) fetal bovine serum (HyClone, Logan, Utah) and 0.25 μM methothrexate (Sigma, St. Louis, Mo.). During selection for stable clones, 700 μg/ml Geneticin® (G418) (Sigma) was added. The non-recombinant CHO-K1 cell line was used as a positive control in the CMP-sialic acid transporter functionality experiment as well as for generation of cDNA template used to amplify CMP-sialic acid transporter cDNA. It was obtained from the American Type Culture Collection (ATCC number CCL-61) (Manassas, Va.). CHO-K1 was maintained in DMEM supplemented with 10% (v/v) fetal bovine serum. The CHO glycosylation mutant, Lec2 was used to test the functionality of the recombinant CMP-sialic acid transporter. It was obtained from the American Type Culture Collection (ATCC number CRL-1736) (Manassas, Va.). Lec2 was isolated through resistance to wheat germ agglutinin, a lectin that binds sialic acid (Stanley & Siminovitch, 1977). The Lec2 cell line was maintained in alpha minimum essential medium (MEM) (Invitrogen) supplemented with 10% (v/v) fetal bovine serum (HyClone). All adherent cells were grown as monolayers in stationary T-flasks and incubated at 37° C. under a 5% CO₂ atmosphere. Cells were detached from T-flasks by adding 0.05% (v/v) trypsin/EDTA solution (Sigma) during regular sub-culturing.

Full Length cDNA Synthesis from Chinese Hamster Ovary Cells (CHO-K1)

Total RNA was isolated with TRIzol® reagent (Invitrogen) as follows. Ten million CHO-K1 cells were harvested from culture and resuspended in 1 ml of TRIzol® reagent. The cells were syringe-sheared 50 times using a 3 ml syringe and a 21 gauge needle. After an incubation of 10 minutes, 200 μl molecular grade chloroform was added and the sample was centrifuged at 14,000 rpm for 15 minutes at 4° C. The upper aqueous layer was then transferred to a new RNAse-free tube and an equal volume of molecular grade isopropanol was added. The tubes were then incubated at −20° C. for 3 hours or more. Following that, a visible pellet was seen after centrifugation at 14,000 rpm for 15 minutes at 4° C. This pellet was washed with 75% (v/v) ethanol, air-dried and dissolved in 35 μl DEPC water. RNA quantification was carried out using the GeneQuant™ Pro RNA/DNA Calculator (Amersham Biosciences, Piscataway, N.J.). RNA quality was assessed using the absorbance ratio of 260 nm to 280 nm, where a ratio of 1.9 and above was considered an indicator of RNA with sufficient purity. Reverse transcription of 10 μg RNA to first strand cDNA was carried out in a 40 μl reaction with 2 μl ImProm-II reverse transcriptase (Promega, Madison, Wis.) and 1 μg oligo dT (Research Biolabs, Singapore) at 42° C. for 1 hour according to manufacturer's instructions. The reaction was terminated at 70° C. for 5 minutes. The cDNA prepared from CHO-K1 mRNA was used as a template for amplification of the CMP-sialic acid transporter cDNA.

### Polymerase Chain Reaction (PCR) Amplification of CMP-Sialic Acid Transporter

The CMP-sialic acid transporter cDNA used for cloning was amplified from CHO-K1 cDNA, based on primers designed from the previously cloned hamster CMP-sialic acid transporter (Eckhardt & Gerardy-Schahn, 1997). Nhe I and EcoR I restriction sites were introduced upstream and downstream of the coding region for subsequent subcloning. The forward primer used was 5′-GAGCTAGCGCCACCATGGCTCAGGCGAG-3′ (SEQ ID NO:12) and the reverse primer used was 5′-TCCGAATTCTCACACACCAATGACTCTTTC-3′ (SEQ ID NO:13), where the introduced restriction sites are underlined, and the incorporated coding regions of the CMP-sialic acid transporter are in bold. All PCR reagents were purchased from Promega (Madison, Wis.) except for the primer stock (Research Biolabs). The 50 μl PCR reaction mix contained 4 μl CHO K1 cDNA template, 1×Pfu buffer, 250 μM dNTP, 2 μM of each primer and a Taq-Pfu polymerase mix of approximately 10 U. Cycling conditions were an initial denaturation of 94° C. for 6 minutes, followed by 30 cycles of 94° C. for 1 minute, 59° C. for 1 minute, 72° C. for 4 minutes, and a final extension of 72° C. for 8 minutes.

### Construction of CMP-Sialic Acid Transporter Expression Vector

The PCR product was first subcloned into pCR®2.1-TOPO from TOPO TA Cloning® according to manufacturer's instructions, and the sequence of the PCR product was verified by comparing it with the previously cloned hamster CMP-sialic acid transporter sequence (GenBank accession number Y12074) and found to be 100% similar. The verified PCR product was then subcloned into pcDNA3.1(+) (Invitrogen) expression vector. A scale up culture of the clone containing the sequence-verified plasmid was then performed and the final plasmid pcDNA-SAT was purified using the QIAfilter Plasmid Maxi (Qiagen, Valencia, Calif.) kit. The concentration of the plasmid was measured using the DU(R) 530 Life Science UV/Vis Spectrophotometer (Beckman Coulter, Fullerton, Calif.) before transfection into CHO IFN-γ.

Generation of CHO IFN-γ Clones with Stable Overexpression of CMP-Sialic Acid Transporter.

To enable the transfection to start from a “pure” cell population, a single cell clone of the parental CHO IFN-γ was isolated through limiting dilution. This single cell clone had similar growth (0.025/hr versus 0.022/hr for single cell clone and parental CHO IFN-γ respectively) and IFN-γ production (2.1×10⁻⁸ μg/cell-hr versus 1.7×10⁻⁸ μg/cell-hr for single cell clone and parental CHO IFN-γ respectively) characteristics when compared to the original parent population. Electroporation was then carried out using the Cell Line Nucleofector Kit T (Amaxa, Gaithersburg, Md.) on the Nucleofector Device (Amaxa). Cells were passaged 2 days before electroporation and nucleofected at 80 to 90% confluency. Approximately 10 μg of linearized plasmids per 1 million cells were used. Four days after transfection, the culture medium was replaced with media containing 700 μg/ml G418 (Sigma) for selection. The G418 selection was maintained for approximately 2 weeks before stable transfected cell pools were obtained, whereas untransfected parent CHO IFN-γ exposed to G418-containing media died within a week. The transfectants were then isolated as single cells through limiting dilution to generate adherent cell lines with stable overexpression of the CMP-sialic acid transporter. A total of 36 single cell clones were screened for CMP-sialic acid transporter overexpression using real-time PCR and Western blot analyses as described below. Four clones were selected for subsequent sialylation analysis of the recombinant IFN-γ product. A set of untransfected parent CHO IFN-γ and cells transfected with null vector pcDNA3.1(+) were also maintained as negative controls. In the latter case, null vector cells were also isolated as single clones and one was randomly chosen for further analysis.

### CMP-Sialic Acid Transporter Functionality Experiment

The expression construct containing CMP-sialic acid transporter, pcDNA-SAT was transfected into Lec2 cells via electroporation as described earlier. Three days after transfection, the cells were analyzed using FACS. Each single cell suspension (1.5 million cells) was incubated in the dark with 20 μg/ml WGA-FITC (Vector Laboratories, Burlingame, Calif.) or PNA-FITC (Vector Laboratories) at room temperature for 15 minutes. Cells were subsequently washed with 1% (w/v) bovine serum albumin (BSA) (Sigma) in PBS before they were analyzed using the FACSCalibur™ System (BD Biosciences, San Jose, Calif.). Results were computed using the accompanying software analysis tools.

### Real-Time PCR

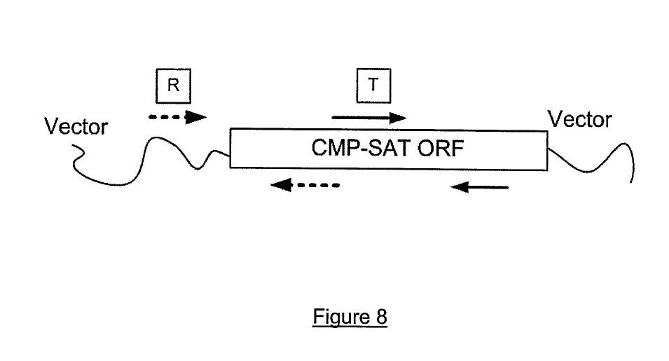

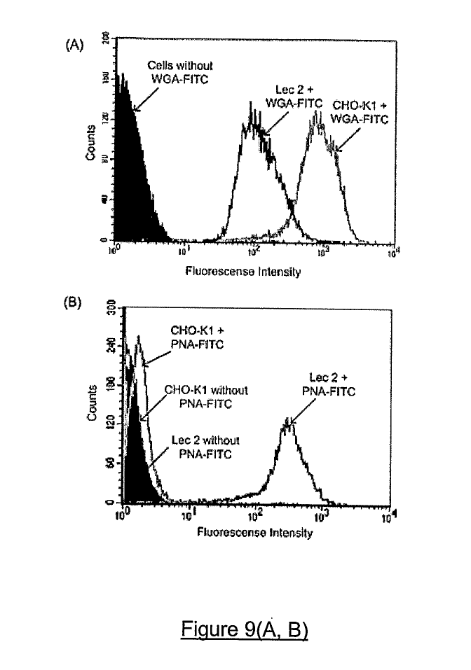

Single strand cDNA was synthesized from stable CHO IFN-γ cell lines as well as the negative controls as was described above and used as template in the real-time PCR reaction. Real-time PCR was carried out using the ABI PRISM® 7000 Sequence Detection System (Applied Biosystems, Foster City, Calif.). The PCR conditions were an initial denaturation of 95° C. for 10 minutes, followed by 40 cycles of 95° C. for 15 seconds and 60° C. for 60 seconds. The reaction buffer of 25 μl 1×SYBR® Green PCR Master Mix (Applied Biosystems) contained 7.5 pmol forward and reverse primers and 1.25 μl of cDNA template. Primers for detection of total and recombinant expression of CMP-sialic acid transporter transcript were designed as shown in FIG. 8. Primers for detection of CHO β-actin was 5′-AGCTGAGAGGGAAATTGTGCG-3′ (SEQ ID NO:14) as the forward primer and 5′-GCAACGGAACCGCTCATT-3′ (SEQ ID NO:15) as the reverse primer. Standard curves were generated simultaneously for each real-time PCR run that was carried out, where serial dilutions of pcDNA-SAT and a CHO β-actin plasmid (courtesy of Dr Peter Morin, Bioprocessing Technology Institute) were used. Both samples and standards were run in duplicate for each run. Using the accompanying software analysis tool, a threshold cycle Ct was defined as the cycle at which a given sample crosses a threshold fluorescence value, where Ct is thus proportional to the amount of starting DNA template. A linear plot of Ct versus the logarithm of plasmid (DNA) concentration was interpolated to find the concentration of the unknown samples. Each total and recombinant CMP-sialic acid transporter concentration was normalized with its respective β-actin concentration, and results from each of the samples from the over expressing clones were compared relative to the normalized concentrations obtained from the untransfected parent CHO IFN-γ sample.

### Antibodies

A peptide (CIQQEATSKERVIGV) (SEQ ID NO:16) corresponding to the C-terminus region of the hamster CMP-sialic acid transporter (SwissProt accession number O08520) was synthesized and rabbit anti-serum against this peptide conjugated with keyhole lympet hemocyanin (KLH) was generated (Open Biosystems, Huntsville, Ala.). Some of the crude serum was also peptide affinity-purified to enrich for CMP-sialic acid transporter polyclonal antibodies (Open Biosystems) and this helped to reduce non-specific binding during Western blot analyses. The anti-rabbit IgG antibody (Jackson ImmunoResearch, West Grove, Pa.) was used for secondary detection. Actin expression was used for normalization and the mouse monoclonal antibody against actin (Abcam, Cambridge, UK) was used with anti-mouse IgG antibody (Abcam) as the secondary antibody.

### SDS-PAGE and Western Blot Analysis

Approximately 10 million cells were collected from stable CHO IFN-γ cell lines as well as the negative controls and washed twice with ice-cold PBS. The cell pellet was lysed and reduced for electrophoresis under conditions described by Eckhardt & Gerardy-Schahn (1997). Equal amounts of protein lysate were prepared using the Coomassie Plus protein assay (Pierce, Rockford, Ill.). Protein lysate samples of 50 μg were loaded onto 12% polyacrylamide gels for SDS-PAGE analysis. The fractionated proteins were then electroblotted onto a PVDF membrane (Biorad, Hercules, Calif.). The membrane was blocked for 1 hour in 5% (w/v) non-fat milk in PBS-T (blocking buffer), followed by an overnight incubation in polyclonal anti-CMP-sialic acid transporter antibody or monoclonal anti-actin diluted 1000 fold in blocking buffer at 4° C. On the next day, membranes are washed in PBS-T before being incubated for 1 hour in anti-rabbit (detection of CMP-sialic acid transporter) or anti-mouse IgG antibody (detection of actin) diluted 10,000 fold in blocking buffer. Membranes were washed in PBS-T before they were detected via chemiluminescense using ECL Western blot detection reagents (Amersham Biosciences) according to the manufacturer's instructions. The protein band intensities were quantified using software analysis tools accompanying the Gel Doc XR system (Biorad). All incubations were at room temperature, unless otherwise stated.

### Glycosylation Analysis of IFNγ

The detailed procedure for glycosylation analysis of IFNγ has been described previously (Wong et al., 2005). Briefly, supernatant from stable CHO IFN-γ cell lines as well as the negative controls were harvested when cells reached confluency in batch cultures. The supernatant was filtered (0.22 μM) and purified through an immunoaffinity column made from purified mouse anti-human IFN-γ clone B27 (BD Pharmigen, San Diego, Calif.). Quantification of IFN-γ was carried out using reverse phase HPLC, where standards of known IFN-γ concentration (Research Diagnostics Inc., Flanders, N.J.) had been run and compared with the actual samples. A Vydac C18 1 mm by 250 mm column was used (Grace Vydac, Hesperia, Calif.) in a Shimadzu LC-10ADvp HPLC system (Shimadzu Corporation, Kyoto, Japan). The sample was eluted over a 30 minute linear gradient from 35% (v/v) to 65% (v/v) buffer B (Buffer A: 0.1% (v/v) trifluoroacetic acid (TFA) in HPLC grade water; buffer B: 0.1% (v/v) in HPLC grade acetonitrile) at a flow rate of 0.06 ml/min. The total sialic acid content of IFN-γ was then measured using a modified version of the thiobarbituric acid assay (Hammond & Papermaster, 1979), after the sialic acid was cleaved from the purified IFN-γ samples using sialidase treatment. Site occupancy of IFN-γ was measured using micellar electrokinetic capillary chromatography.

### Results

### Testing the Functionality of Recombinant CMP-Sialic Acid Transporter (CMP-SAT)

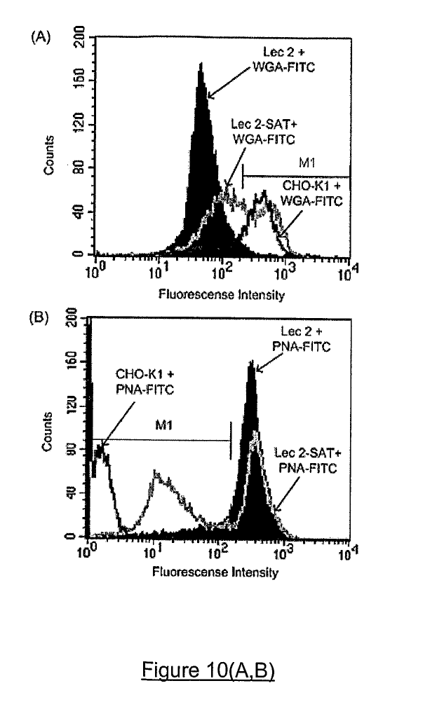

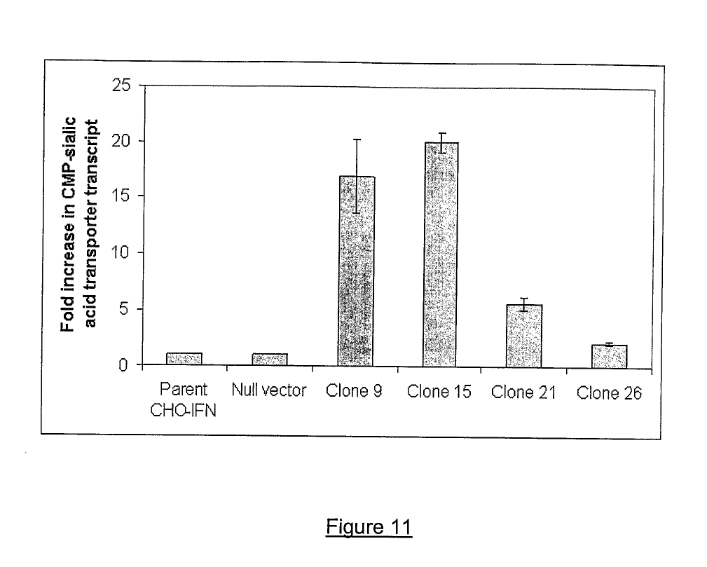

The model cell line CHO IFN-γ contains endogenous CMP-sialic acid transporter. It was necessary to ensure that transfection of the CMP-sialic acid transporter expression construct would result in expression of functional recombinant CMP-sialic acid transporter and that transporter activity was not just due to the endogenous CMP-sialic acid transporter. As mentioned earlier, the Lec2 cell line is a CHO mutant cell line that is unable to sialylate glycoproteins due to a CMP-sialic acid transporter defect (Deutscher et al., 1984). Transfection of a functional CMP-sialic acid transporter will correct the defect and result in sialylated glycoproteins. An assay to test the CMP-sialic acid transporter construct could thus be designed based on the characteristics of this cell line. Lectins were used to detect the difference in Lec2 surface glycoproteins before and after transfection of the CMP-sialic acid transporter expression construct. Wheat-germ agglutinin (WGA) and peanut agglutinin (PNA) conjugated to fluorescein-isothiocyanate (FITC), which binds to sialic acid (Ishida et al., 1998) and galactose (Aoki et al., 2001) respectively, were used in a FACS analysis. To demonstrate that the lectins had the ability to differentiate between sialylated and non-sialylated glycoproteins for this experiment, surface glycoproteins produced by CHO-K1 and Lec2 cells were detected using WGA-FITC and PNA-FITC in a FACS analysis (FIG. 9A,B). As expected, Lec2 surface glycoproteins had less binding with WGA-FITC (FIG. 9A) and more binding with PNA-FITC (FIG. 9B) as compared to CHO-K1 surface glycoproteins, as demonstrated by the relative detection of cell populations with lower and higher fluorescence respectively. Transient transfection of pcDNA-SAT into Lec2 cells resulted in a bimodal distribution of the cells with one sub-population shifting to the right when WGA-FITC was used for the FACS analysis (FIG. 10A). This demonstrated the binding of sialylated glycoproteins on that sub-population of Lec2 cells where the recombinant CMP-sialic acid transporter was able to correct the mutant defect in these cells. These results were confirmed when a converse effect was seen with the PNA-FITC (galactose-binding lectin) FACS analysis (FIG. 10B). This demonstrated the functionality of the CMP-sialic acid transporter expressed from pcDNA-SAT, which was able to correct the defect in Lec2 cells. The results obtained concur with previously reported analyses on the ability of the CMP-sialic acid transporter to correct the defect in Lec2 glycosylation mutants (Aoki et al., 2001; Ishida et al., 1998). Thus, a similar transfection of pcDNA-SAT into CHO IFN-γ would lead to overexpression of functional CMP-sialic acid transporter.

### Detection of Over Expressed CMP-Sialic Acid Transporter in Stable Cell Lines

Stable integration of expression vectors into the host chromosome occurs almost purely by non-homologous recombination and its integration sites are randomly distributed (Kaufman, 1990). As with other cell engineering approaches, it was thus necessary to screen the CHO IFN-γ sub-clones for CMP-sialic acid transporter overexpression. Overexpression of CMP-sialic acid transporter was detected at the transcript level using real-time PCR and at the protein level using Western blot analysis. Real-time PCR analysis was chosen to compare CMP-sialic acid transporter transcript levels since it has proven to be a useful assay for detection of low abundance mRNA (Bustin, 2000). It has a wide dynamic range of quantification of 7 to 8 logarithmic decades, a high technical sensitivity and a high precision (Klein, 2002). In addition, due to the specificity of the primers in amplifying selected gene regions, it allowed comparison of total and recombinant expression of CMP-sialic acid transporter (FIG. 8) (SEQ ID NOS:8-11).

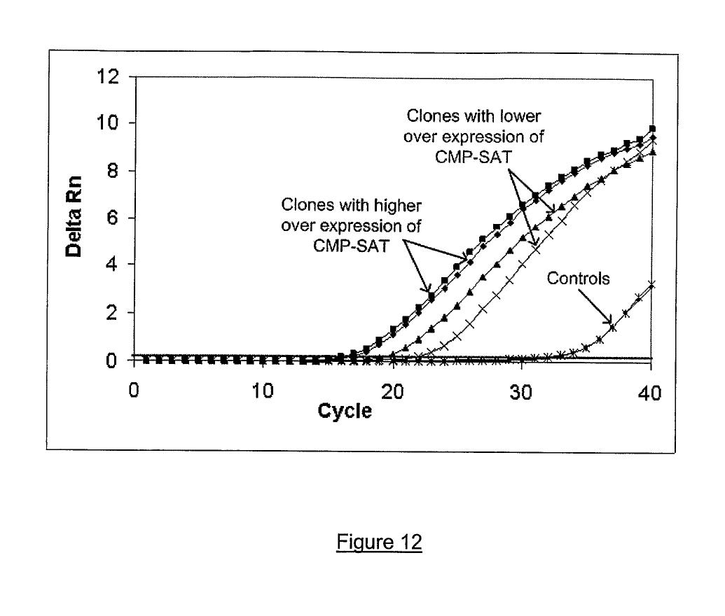

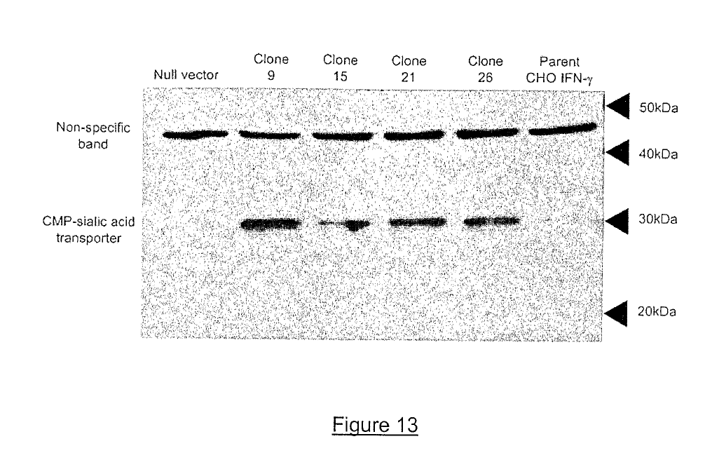

The 4 clones over expressing CMP-sialic acid transporter showed 2 to 20 fold increase in total CMP-sialic acid transporter transcript as compared to the untransfected parent CHO IFN-γ sample (FIG. 11). This could be attributed to the recombinant CMP-sialic acid transporter expression in these clones since a corresponding increase in recombinant transporter transcript was observed (FIG. 12). This showed that the 4 clones isolated had higher CMP-sialic acid transporter expression due to the vector transfection and it was not just due to the selection of random clones that had higher endogenous CMP-sialic acid transporter expression. Overexpression of the CMP-sialic acid transporter was also detected at the protein level using Western blot analyses (FIG. 13).

The fold increase in total CMP-sialic acid transporter transcript (FIG. 11) generally resulted in increased transporter protein expression (FIG. 13) which would be expected if no regulation existed during the processes of CMP-sialic acid transporter transcription and translation. However, the range of fold increase in transporter transcript levels (approximately 2 to 20 fold) did not result in a similar range of fold increase in transporter protein overexpression (approximately 1.8 to 2.8 fold as shown in Table 2). It is established that transcript increase is not always a good indicator of protein expression and hence the need to profile the expression at both levels (Cox et al., 2005). Moreover, the existence of post-translational modifications adds further complexity, since proteins that are successfully translated do not necessarily mature to functional form. Finally, a functional protein must also be targeted to the correct location before it can perform its role in the cell.

One of the postulated reasons for this lack of correlation between transcript and protein levels of CMP-sialic acid transporter overexpression is the saturation in the expression of the transporter protein by the cells. Though the CMP-sialic acid transporter is a relatively small protein of approximately 30 kDa, it is a transmembrane protein (Eckhardt and Gerady-Schahn, 1997) which needs to be correctly folded, inserted with the correct membrane topology and localized at the trans-Golgi membrane before it can functionally transport CMP-sialic acid. As the CMP-sialic acid transporter contains 10 transmembrane spanning helices (Eckhardt et al., 1999), its correct insertion would probably depend on internal hydrophobic topogenic sequences, as with other multipass transmembrane proteins (Lodish et al., 2000). The targeting and localization of CMP-sialic acid transporter seems to be determined by specific stretches of amino acids within the open reading frame of the protein sequence (Eckhardt et al., 1998). Since the recombinant CMP-sialic acid transporter was cloned from CHO-K1 cDNA containing endogenous CMP-sialic acid transporter, it would thus possess the above characteristics for correct membrane insertion and trans-Golgi localization. Nevertheless, it is postulated that the heterologous overexpression of the CMP-sialic acid transporter could still affect the efficiency of this process. For example, mis-folding in the ER would result in the targeting of the recombinant CMP-sialic acid transporter for degradation instead of its retention as functional protein. This would result in the detection of a lower range of fold increase in transporter protein overexpression as compared to fold increase in transcript expression. However, it was eventually more important to consider whether the current levels of CMP-sialic acid transporter overexpression was sufficient to improve sialylation of the recombinant protein product IFN-γ, which is what that will be presented next.

### Effects of CMP-Sialic Acid Transporter Overexpression on IFNγ Glycosylation

Two quantitative indices were used to compare the extent of sialylation of recombinant IFN-γ between the clones over expressing CMP-sialic acid transporter and the negative controls, untransfected parent CHO IFN-γ and null vector cell line. The IFN-γ sialic acid content (Eq. 2) measures the average amount of sialic acid on the protein. If IFN-γ is assumed to have complete site occupancy and only biantennary glycoforms, a theoretical maximum of 4 molecules (or moles) of sialic acid per molecule (or mole) of IFNγ can be calculated for this glycoprotein.

$$IFN γ sialic acid content = \frac{Moles of sialic acid}{Moles of IFN γ} \qquad ( 2 )$$

However, the site occupancy of IFNγ is never 100%. Thus, a more direct indicator of sialylation extent is site sialylation. This index normalizes the sialic acid on IFNγ to the available N-linked sites (Eq. 3) This normalization allows the direct consideration of the cells' ability to sialylate a given site. Thus, it becomes a direct measure of the effectiveness of CMP-sialic acid transporter overexpression in improving the sialylation process in the cells.

$$IFN γ site sialylation = \frac{IFN γ sialic acid content}{0.01 [ 2 ( \% 2 N ) + 1 ( \%1 N ) + 0 ( \%0 N ) ]} \qquad ( 3 )$$

where %2N, %1N and %0N is the percentage of 2-site, 1-site and 0-site glycosylated IFNγ respectively. Since CMP-sialic acid transporter overexpression affects sialylation, the site occupancy of IFN-γ is not expected to vary since the process of oligosaccharide transfer onto the protein is upstream to the sialylation process. Thus, by using average site occupancy values of 78.5%2N, 18.0% 1N and 3.5% 0N (Gu, 1997), the actual maximum sialic acid content of IFNγ is calculated to be 3.5 moles of sialic acid per mole of IFN-γ. This becomes the maximum IFN-γ sialic acid content that can be achieved in CHO IFN-γ.

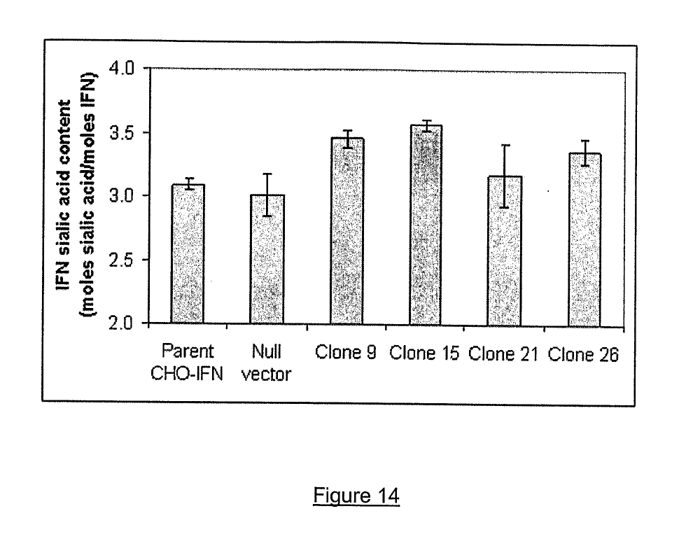

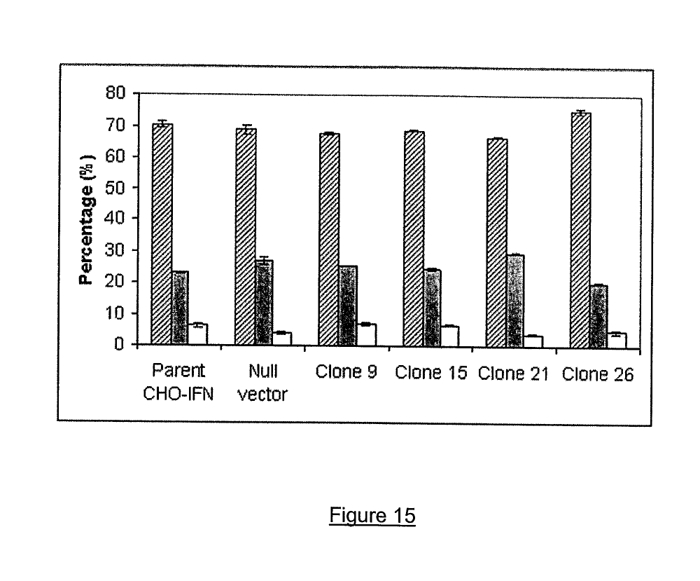

Adherent batch cultures were performed using the same 4 single cell clones over expressing CMP-sialic acid transporter and the negative controls. Each of the 4 clones exhibited an increase in IFN-γ sialic acid content when compared to the negative controls (FIG. 14). As expected, the IFN-γ site occupancy was not affected significantly by CMP-sialic acid transporter overexpression (FIG. 15). The extent of overexpression of CMP-sialic acid transporter was compared with the normalized IFN-γ sialic acid content in terms of site sialylation (Table 2). There was 4 to 16% relative increase in IFN-γ site sialylation of the single cell clones over expressing CMP-sialic acid transporter when compared to untransfected parent CHO IFN-γ. Clones 9 and 15 which had a higher fold increase in CMP-sialic acid transporter protein levels was observed to have a corresponding greater improvement in IFN-γ site sialylation as compared to clones 21 and 26 (Table 2). Through the trends observed in these 4 clones, it can be more conclusively inferred that it was the CMP-sialic acid transporter overexpression which resulted in the increase in IFN-γ site sialylation. When the IFN-γ sialic acid content of clones 9 and 15 (Table 2) was considered in the context of the actual maximum of 3.5 moles of sialic acid per mole of IFN-γ that was mentioned earlier, these clones are producing recombinant IFN-γ with maximal sialylation. These results thus verify the hypothesis that overexpression of CMP-sialic acid transporter would lead to increased sialylation in Chinese hamster ovary cells.

TABLE 2
Summary of extent of CMP-sialic acid transporter (CMP-SAT) overexpression
and IFN-γ site sialylation in adherent stable cell lines.

| | | | Site sialylation | | |
|---|---|---|---|---|---|
| | | | of IFN-γ | Relative | Fold increase |
| | IFN-γ sialic | Number of | (molecules | percentage | in CMP-SAT |
| | acid content | glycans per | sialic acid/site) | increase in site | protein |
| Cell line | (mol/mol)^{a} | IFN-γ^{b} | (Eq. 2) | sialylation^{c} (%) | expression^{d} |
| Parent | 3.09 ± 0.04 | 1.64 | 1.89 | | 1.0 |
| CHO IFN-γ | | | | | |
| Null vector | 3.01 ± 0.16 | 1.65 | 1.82 | −3.7 | 0.93 |
| Clone 9 | 3.45 ± 0.07 | 1.61 | 2.14 | 13.2 | 2.78 |
| Clone 15 | 3.57 ± 0.04 | 1.62 | 2.20 | 16.4 | 1.81 |
| Clone 21 | 3.17 ± 0.24 | 1.62 | 1.96 | 3.7 | 1.73 |
| Clone 26 | 3.36 ± 0.10 | 1.70 | 1.98 | 4.8 | 1.78 |

^{a}IFN-γ sialic acid content is the average sialic acid content of IFN-γ, as plotted in FIG. 14.
^{b}The number of glycans was calculated using IFN-γ site occupancy measurements based on the formula shown on the denominator of Eq. 3.
^{c}The percentage increase in IFN-γ site sialylation was computed relative to the IFN-γ site sialylation of untransfected parent CHO IFN-γ
^{d}The relative expression of CMP-SAT protein was determined by densitometry analysis of Western blots (FIG. 13). Each sample was normalized with its corresponding β-actin expression and compared relative to the normalized CMP-SAT expression of untransfected parent CHO IFN-γ.

TABLE 3
Summary of sialylation improvement strategies that have been previously reported.

| | Cell line/Recombinant | | Percentage increase |
|---|---|---|---|
| Reference | protein of interest | Approach | in sialylation (%) |
| Chitlaru et al. | HEK 293/human | Over express α2,6- | 62% ¹ |
| (1998) | acetylcholinesterase | sialyltransferase (α2,6- | |
| | | ST) | |
| Gu & Wang | CHO/IFN-γ | N-acetylmannosamine | 15% ² |
| (1998) | | (ManNAc) feeding | |
| Weikert et al. | CHO/TNFR-IgG | Over express α2,3- | 30% ¹ |
| (1999) | | sialyltransferase | |
| | | (α2,3-ST) | |
| Bragonzi | CHO/IFN-γ | Over express α2,6-ST | 4% ¹ |
| et al. (2000) | | | |
| Fukuta | CHO/IFN-γ | Over express α2,3 and/or | Up to 23% ² |
| et al. (2000) | | α2,6-ST | |
| Jassal et al. (2001) | CHO/anti-NIP IgG3 | Over express α2,6-ST | 11% ¹ |
| Baker et al. (2001) | CHO/TIMP-I | ManNAc feeding | Marginal (0.8%) ² |
| Baker et al. (2001) | NS0/TIMP-I | ManNAc feeding | Marginal (1.0%) ² |

¹ Percentage increase in sialylation was computed based on sialylation indices similar to IFN-γ sialic acid content (Eq. 2).
² Percentage increase in sialylation was computed based on sialylation indices similar to IFN-γ site sialylation, which takes into account actual number of available sites for sialylation (Eq. 3).

### REFERENCES

Aoki K, Ishida N, Kawakita M. 2001. Substrate recognition by UDP-galactose and CMP-sialic acid transporters. J Biol. Chem. 274:21555-21561.
Andersen D C, Krummen L. 2002. Recombinant protein expression for therapeutic applications. Curr Opin Biotechnol 13:117-123.
Apweiler R, Hermjakob H, Sharon N. 1999. On the frequency of N-glycosylation as deduced from analysis of the SWISS-PROT database. Biochimica et Biophysica Acta 1473:4-8.
Aumiller J J and Jarvis D L. Expression and functional characterization of a nucleotide sugar transporter from Drosophila melanogaster: relevance to protein glycosylation in insect cell expression systems, Protein Expr. Purif. 26 (3), 438-448 (2002).
Bailey J E, Prati E G P, Jean-Mairet J, Sburlati A R, Umaña P. 1998. Engineering glycosylation in animal cells. In Merten O-M, Perrin P, Griffiths B, editors. New developments and new applications in animal cell technology. Boston: Kluwer Academic. pp 5-23.
Baker K N, Rendall M H, Hills A E, Hoare M, Freedman R B, James D C. 2001. Metabolic control of recombinant protein N-glycan processing in NS0 and CHO cells. Biotechnol Bioeng. 73:188-202.
Berninsone P, Eckhardt M, Gerardy-Schahn R, Hirschberg C B. 1997. Functional expression of the murine Golgi CMP-sialic acid transporter in Saccharomyces cerevisiae, J Biol. Chem. 272:12616-12619.
Berninsone P, Hirschberg C B. 2000. Nucleotide sugar transporters of the Golgi apparatus, Curr Opin Struct Biol. 10:542-547.
Bragonzi A, Distenfano G, Buckberry L D, Acerbis G, Foglieni C, Lamotte D, Campi G, Marc A, Soria M R, Jenkins N, Monaco L. 2000. A new Chinese hamster ovary cell line expressing α2,6-sialyltransferase used as a universal host for the production of human-like sialylated recombinant glycoproteins. Biochimica et Biophysica Acta 1474:273-282.
Bustin S A. 2000. Absolute quantification of mRNA using real time reverse-transcription polymerase chain reaction assays. J Mol. Endocrinol. 25:169-193.
Chitlaru T, Kronman C, Zeevi M, Kam M, Harel A, Ordentlich A, Velan B, Shafferman A. 1998. Modulation of circulatory residence of recombinant acetylcholinesterase through biochemical or genetic manipulation of sialylation levels. Biochem J. 336:647-658.
Cox B, Kislinger T, Emili A. 2005. Integrating gene and protein expression data: pattern analysis and profile mining. Methods 35:303-314.
Deutscher S L, Nuwayhid N, Stanley P, Briles E I B, Hirschberg C B. 1984. Translocation across Golgi vesicle membranes: A CHO glycosylation mutant deficient in CMP-sialic acid transport. Cell 39:295-299.
Eckhardt M, Gerardy-Schahn R. 1997. Molecular cloning of the hamster CMP-sialic acid transporter. Eur J. Biochem. 248:187-192.
Eckhardt M, Gotza B, Gerardy-Schahn R. 1998. Mutants of the CMP-sialic acid transporter causing the Lec2 phenotype. J Biol. Chem. 273:20189-20195.
Eckhardt M, Gotza B, Gerardy-Schahn R. 1999. Membrane topology of the mammalian CMP-sialic acid transporter. J Biol. Chem. 274:8779-8787.
Farrar M A, Schreiber RD. 1993. The molecular cell biology of interferon-γ and its receptor, Annu Rev Immunol. 11:571-611.
Ferrari J, Gunson J, Lofgren J, Krummen L. 1998. Chinese hamster ovary cells with constitutively expressed sialidase antisense RNA produce recombinant DNAse in batch culture with increased sialic acid. Biotechnol Bioeng. 60:589-595.
Fukuta K, Yokomatsu T, Abe R, Asanagi M, Makino T. 2000. Genetic engineering of CHO cells producing human interferon-γ by transfection of sialyltransferases. Glycoconj J. 17:895-904.
Grabenhorst E, Schlenke P, Pohl S, Nimtz M, Conradt H S. 1998. Genetic engineering of recombinant glycoproteins and the glycosylation pathway in mammalian host cells. Glycoconj J. 16:81-97.
Gu X. 1997. Characterization and improvement of interferon-γ glycosylation in Chinese hamster ovary cell culture. PhD thesis. MIT. Cambridge Mass. USA.
Gu X, Harmon B J, Wang D I C. 1997. Site and branch-specific sialylation of recombinant human interferon-γ in Chinese hamster ovary cell culture. Biotechnol Bioeng. 55:390-398.
Gu X, Wang D I C. 1998. Improvement of interferon-γ sialylation in Chinese hamster ovary cell culture by feeding N-acetylmannosamine. Biotechnol Bioeng. 58:642-648. Hammond K S, Papermaster D S. 1979. Fluorometric assay of sialic acid in the picomole range: a modification of the thiobarbituric acid assay. Anal Biochem. 74:292-297.
Hills A E, Patel A, Boyd P, James D C. 2001. Metabolic control of recombinant monoclonal antibody N-glycosylation in GS-NS0 cells. Biotechnol Bioeng. 75:239-251.
Hirschberg C B, Robbins P W, Abeijon C. 1998. Transporters of nucleotide sugars, ATP, and nucleotide sulfate in the endoplasmic reticulum and Golgi apparatus. Annu Rev Biochem. 67:49-69.
Hirschberg C B, Snider M D. 1987. Topography of glycosylation in the rough endoplasmic reticulum and Golgi apparatus. Annu Rev Biochem. 56:63-87.
Hooker A D, Goldman M H, Markham N H, James D C, Ison A P, Bull A T, Strange P G, Salmon I, Baines A J, Jenkins N. 1995. N-glycans of recombinant human interferon-γ change during batch culture of Chinese hamster ovary cells. Biotechnol Bioeng. 48:638-648.
Hooker A D, Green N H, Baines A J, Bull A T, Jenkins N, Strange P G, James D C. 1999. Constraints on the transport and glycosylation of recombinant IFN-γ in Chinese hamster ovary and insect cells. Biotechnol Bioeng. 63:559-572.
Ishida N, Ito M, Yoshioka S, Sun-Wada G, Kawakita M. 1998. Functional expression of human Golgi CMP-sialic acid transporter in the Golgi complex of a transport-deficient Chinese hamster ovary cell mutant. J Biochem 124:171-178.
Jassal R, Jenkins N, Charlwood J, Camilleri P, Jefferis R, Lund J. 2001. Sialylation of human IgG-Fc carbohydrate by transfected rat α2,6-sialyltransferase. Biochem Biophy Res Commun. 286:243-249.
Jenkins N, Curling E M A. (1994) Glycosylation of recombinant proteins: problems and prospects. Enzyme Microb Technol. 16:354-364.
Kaufman RJ. 1990. Use of recombinant DNA technology for engineering mammalian cells to produce proteins. In Lubiniecki A S, editor. Large-scale Mammalian Cell Culture Technology. New York: Marcel Dekker. pp. 15-70.
Kawakita M, Ishida N, Miura N, Sun-Wada G, Yoshioka S. 1998. Nucleotide sugar transporters: Elucidation of their molecular identity and its implications for further studies. J Biochem 123:777-785.
Keyt B A, Paoni N F, Refino C J, Berleau L, Nguyen H, Chow A, Lai J, Peña L, Pater C, Ogez J, Etcheverry T, Botstein D, Bennett W F. 1994. A faster-acting and more potent form of tissue plasminogen activator Proc Natl Acad Sci USA. 91:3670-3674.
Kim K, Lawrence S M, Park J, Pitts L, Vann W F, Betenbaugh M J, Palter K B. Expression of a functional Drosophila melanogaster N-acetylneuraminic acid (Neu5Ac) phosphate synthase gene: evidence for endogenous sialic acid biosynthetic ability in insects, Glycobiology 12(2), 73-83 (2002).
Klein D. 2002. Quantification using real-time PCR technology: application and limitations, Trends Mol. Med. 8:257-260.
Kornfeld R, Kornfeld S. 1985. Assembly of asparagines-linked oligosaccharides. Annu Rev Biochem. 54:631-664.
Koury M J. 2003. Sugar coating extends half-lives and improves effectiveness of cytokine hormones. Trends Biotechnol. 21:462-464.
Lee E U, Roth J, Paulson J C. 1989. Alteration of terminal glycosylation sequences on N-linked oligosaccharides of Chinese hamster ovary cells by expression of β-galactosidase α2,6-sialyltransferase. J Biol. Chem. 264:13848-13855.
Liu DT. 1992. Glycoprotein pharmaceuticals: scientific and regulatory considerations, and the US Orphan Drug Act. Trends Biotechnol. 10:114-120.
Lodish H, Berk A, Zipursky S L, Matsudaira P, Baltimore D, Darnell J. 2000. Molecular Cell Biology. New York: W.H. Freeman and Company. pp. 691-745.
Minch S L, Kallio P T, Bailey J E. 1995. Tissue plasminogen activator coexpressed in Chinese hamster ovary cells with α(2,6)-sialyltransferase contains NeuAcα(2,6)Galβ(1,4)Glc-N-AcR linkages. Biotechnol Prog. 11:348-351.
Pels Rijckens W B, Overdijk B, Van Den Eijnden D H, Ferwerda W. 1995. The effects of increasing nucleotide-sugar concentrations on the incorporation of sugars into glycoconjugates in rat hepatocytes. Biochem J. 305:865-870.
Sburlati A R, Umaña P, Prati E G P, Bailey J E. 1998. Synthesis of bisected glycoforms of recombinant IFN-β by overexpression of β1,4-N-acteylglucosaminyltransferase III in Chinese hamster ovary cells. Biotechnol Prog. 14:189-192.
Scahill S J, Devos J, Van der Heyden J, Fiers W. 1983. Expression and characterization of the product of a human immune interferon cDNA gene in Chinese hamster ovary cells, Proc Natl Acad Sci USA 80:4654-4658.
Segawa H, Kawakita M and Ishida N. Human and Drosophila UDP-galactose transporters transport UDP-N-acetylgalactosamine in addition to UDP-galactose, Eur. J. Biochem. 269 (1), 128-138 (2002).
Stanley P, Siminovitch L. 1977. Complementation between mutants of CHO cells resistant to a variety of plant lectins. Somatic Cell Genet. 3:391-405.
Varki A. 1993. Biological roles of oligosaccharides: all of the theories are correct. Glycobiology 3:97-130.
Warner T G. 1999. Enhancing therapeutic glycoprotein production in Chinese hamster ovary cells by metabolic engineering endogenous gene control with antisense DNA and gene targeting. Glycobiology 9(9):841-850.
Weikert S, Papac D, Briggs J, Cowfer D, Tom S, Gawlitzek M, Logfren J, Mehta S, Chrisholm V, Modi N, Eppler S, Carroll K, Chamow S, Peers D, Berman P, Krummen L. 1999. Engineering Chinese hamster ovary cells to maximize sialic acid content of recombinant glycoproteins. Nat. Biotechnol. 17:1116-1121.
Weiss P, Ashwell G. 1989. The asialoglycoprotein receptor: Properties and modulation by ligand. Prog Clin Biol Res. 300:169-184.
Wong D C F, Wong K T K, Goh L T, Heng C K, Yap M G S. 2005. Impact of dynamic online fed-batch strategies on metabolism, productivity and N-glycosylation quality in CHO cell cultures. Biotechnol Bioeng. 89:164-177.

1. A mammalian cell producing a CMP-SAT, a fragment or a variant thereof, at above endogenous levels, and producing heterologous glycoprotein(s) with sialylation at above endogenous level.

2. The mammalian cell of claim 1, wherein the cell produces CMP-SAT at above endogenous levels and produces heterologous glycoprotein(s) with sialylation at above endogenous level when compared to a naturally occurring cell.

3. The mammalian cell of claim 1, wherein the cell is a Chinese Hamster Ovary (CHO) cell.

4. The mammalian cell of claim 1, wherein the cell is a human cell.

5. The mammalian cell of claim 1, wherein the cell is transformed with a gene encoding CMP-SAT or a construct comprising that gene.

6. The mammalian cell of claim 5, wherein the cell before transformation with a gene encoding CMP-SAT is a naturally occurring cell for the expression of CMP-SAT.

7. The mammalian cell of claim 1, wherein the CMP-SAT gene is CHO or human gene.

8. The mammalian cell of claim 1, wherein the glycoprotein is mammalian.

9. The mammalian cell of claim 1, wherein the glycoprotein is human.

10. The mammalian cell of claim 1, wherein the glycoprotein is any IFN-γ, a fragment or a variant thereof.

11. The mammalian cell of claim 1, wherein the cell is transformed with a gene encoding a heterologous protein or with a construct comprising that gene.

12. The mammalian cell of claim 1, wherein the cell is transformed with a gene encoding at least a IFN-γ, a fragment or a variant thereof.

13. The mammalian cell of claim 1, wherein the cell is a CHO cell, and the cell produces CMP-SAT, a fragment or a variant thereof, at above endogenous levels, and wherein the cell produces sialylation of at least one glycoprotein at above endogenous level.

14. The mammalian cell of claim 1, which is in the form of an isolated cell line.

15. The mammalian cell of claim 1, wherein the mammalian cell is included in a kit for the expression of at least one sialylated glycoprotein.

16. A method for the preparation of heterologous sialylated glycoprotein(s) in a cell or cell line comprising enhancing the expression of a CMP-SAT, a fragment or a variant thereof, at above endogenous levels, and enhancing the sialylation of heterologous glycoprotein(s) at above endogenous level.

17. The method of claim 16, wherein CMP-SAT is expressed at above endogenous levels and produces heterologous glycoprotein(s) with sialylation at above endogenous level when compared to a naturally occurring cell or cell line.

18. The method of claim 16, wherein the cell or cell line is Chinese Hamster Ovary (CHO) or human.

19. The method of claim 16, wherein the sialylated glycoprotein(s) is a heterologous mammalian glycoprotein.

20. The method of claim 16, wherein the sialylated glycoprotein(s) is any IFN-γ, a fragment or a variant thereof.

21. The method of claim 16, wherein the cell or cell before transformation with a gene encoding CMP-SAT is a naturally occurring cell or cell line for the expression of CMP-SAT.

22. A method for producing at least a heterologous sialylated glycoprotein in a mammalian cell or cell line comprising:
(a) transforming a mammalian cell with a gene or a construct comprising said gene encoding a CMP-SAT, a fragment or a variant thereof, at above endogenous levels; and
(b) transforming the cell with an heterologous gene or with a construct comprising said gene encoding at least a glycoprotein of interest sialylated at above endogenous level.

23. The method of claim 22, wherein the cell is transformed with a heterologous gene or construct comprising said gene encoding a CMP-SAT, a fragment or a variant thereof, at above endogenous levels and encoding at least a glycoprotein of interest sialylated at above endogenous level when compared to a naturally occurring cell.

24. The method of claim 22, wherein the mammalian cell before transformation with a gene encoding CMP-SAT is a naturally occurring cell for the expression of CMP-SAT.

25. The method of claim 22, wherein the at least a heterologous sialylated glycoprotein is at least an IFN-γ, a fragment or a variant thereof.
